SEQUENCE LISTING (13 sequences)

SEQ ID NO: 1 - protein, 394 aa, Homo sapiens
MATAQLQRTPMSALVFPNKISTEHQSLVLVKRLLAVSVSCITYLRGIFPECAYGTRYLDDLCVKILREDKNCPGSTQLVKWMLGCYDALQKKYLRMVVLAVYTNPEDPQTISECYQFKFKYTNNGPLMDFISKNQSNESSMLSTDTKKASILLIRKIYILMQNLGPLPNDVCLTMKLFYYDEVTPPDYQPPGFKDGDCEGVIFEGEPMYLNVGEVSTPFHIFKVKVTTERERMENIDSTILSPKQIKTPFQKILRDKDVEDEQEHYTSDDLDIETKMEEQEKNPASSELEEPSLVCEEDEIMRSKESPDLSISHSQVEQLVNKTSELDMSESKTRSGKVFQNKMANGNQPVKSSKENRKRSQHESGRIVLHHFDSSSQESVPKRRKFSEPKEHI

SEQ ID NO: 2 - protein, 3130 aa, Homo sapiens
MFSVRIVTADYYMASPLQGLDTCQSPLTQAPVKKVPVVRVFGATPAGQKTCLHLHGIFPYLYVPYDGYGQQPESYLSQMAFSIDRALNVALGNPSSTAQHVFKVSLVSGMPFYGYHEKERHFMKIYLYNPTMVKRICELLQSGAIMNKFYQPHEAHIPYLLQLFIDYNLYGMNLINLAAVKFRKARRKSNTLHATGSCKNHLSGNSLADTLFRWEQDEIPSSLILEGVEPQSTCELEVDAVAADILNRLDIEAQIGGNPGLQAIWEDEKQRRRNRNETSQMSQPESQDHRFVPATESEKKFQKRLQEILKQNDFSVTLSGSVDYSDGSQEFSAELTLHSEVLSPEMLQCTPANMVEVHKDKESSKGHTRHKVEEALINEEAILNLMENSQTFQPLTQRLSESPVFMDSSPDEALVHLLAGLESDGYRGERNRMPSPCRSFGNNKYPQNSDDEENEPQIEKEEMELSLVMSQRWDSNIEEHCAKKRSLCRNTHRSSTEDDDSSSGEEMEWSDNSLLLASLSIPQLDGTADENSDNPLNNENSRTHSSVIATSKLSVKPSIFHKDAATLEPSSSAKITFQCKHTSALSSHVLNKEDLIEDLSQTNKNTEKGLDNSVTSFTNESTYSMKYPGSLSSTVHSENSHKENSKKEILPVSSCESSIFDYEEDIPSVTRQVPSRKYTNIRKIEKDSPFIHMHRHPNENTLGKNSFNFSDLNHSKNKVSSEGNEKGNSTALSSLFPSSFTENCELLSCSGENRTMVHSLNSTADESGLNKLKIRYEEFQEHKTEKPSLSQQAAHYMFFPSVVLSNCLTRPQKLSPVTYKLQPGNKPSRLKLNKRKLAGHQETSTKSSETGSTKDNFIQNNPCNSNPEKDNALASDLTKTTRGAFENKTPTDGFIDCHFGDGTLETEQSFGLYGNKYTLRAKRKVNYETEDSESSFVTHNSKISLPHPMEIGESLDGTLKSRKRRKMSKKLPPVIIKYIIINRFRGRKNMLVKLGKIDSKEKQVILTEEKMELYKKLAPLKDFWPKVPDSPATKYPIYPLTPKKSHRRKSKHKSAKKKTGKQQRTNNENIKRTLSFRKKRSHAILSPPSPSYNAETEDCDLNYSDVMSKLGFLSERSTSPINSSPPRCWSPTDPRAEEIMAAAEKEAMLFKGPNVYKKTVNSRIGKTSRARAQIKKSKAKLANPSIVTKKRNKRNQTNKLVDDGKKKPRAKQKTNEKGTSRKHTTLKDEKIKSQSGAEVKFVLKHQNVSEFASSSGGSQLLFKQKDMPLMGSAVDHPLSASLPTGINAQQKLSGCFSSFLESKKSVDLQTFPSSRDDLHPSVVCNSIGPGVSKINVQRPHNQSAMFTLKESTLIQKNIFDLSNHLSQVAQNTQISSGMSSKIEDNANNIQRNYLSSIGKLSEYRNSLESKLDQAYTPNFLHCKDSQQQIVCIAEQSKHSETCSPGNTASEESQMPNNCFVTSLRSPIKQIAWEQKQRGFILDMSNFKPERVKPRSLSEAISQTKALSQCKNRNVSTPSAFGEGQSGLAVLKELLQKRQQKAQNANTTQDPLSNKHQPNKNISGSLEHNKANKRTRSVTSPRKPRTPRSTKQKEKIPKLLKVDSLNLQNSSQLDNSVSDDSPIFFSDPGFESCYSLEDSLSPEHNYNFDINTIGQTGFCSFYSGSQFVPADQNLPQKFLSDAVQDLFPGQAIEKNEFLSHDNQKCDEDKHHTTDSASWIRSGTLSPEIFEKSTIDSNENRRHNQWKNSFHPLTTRSNSIMDSFCVQQAEDCLSEKSRLNRSSVSKEVFLSLPQPNNSDWIQGHTRKEMGQSLDSANTSFTAILSSPDGELVDVACEDLELYVSRNNDMLTPTPDSSPRSTSSPSQSKNGSFTPRTANILKPLMSPPSREEIMATLLDHDLSETIYQEPFCSNPSDVPEKPREIGGRLLMVETRLANDLAEFEGDFSLEGLRLWKTAFSAMTQNPRPGSPLRSGQGVVNKGSSNSPKMVEDKKIVIMPCKCAPSRQLVQVWLQAKEEYERSKKLPKTKPTGVVKSAENFSSSVNPDDKPVVPPKMDVSPCILPTTAHTKEDVDNSQIALQAPTTGCSQTASESQMLPPVASASDPEKDEDDDDNYYISYSSPDSPVIPPWQQPISPDSKALNGDDRPSSPVEELPSLAFENFLKPIKDGIQKSPCSEPQEPLVISPINTRARTGKCESLCFHSTPIIQRKLLERLPEAPGLSPLSTEPKTQKLSNKKGSNTDTLRRVLLTQAKNQFAAVNTPQKETSQIDGPSLNNTYGFKVSIQNLQEAKALHEIQNLTLISVELHARTRRDLEPDPEFDPICALFYCISSDTPLPDTEKTELTGVIVIDKDKTVFSQDIRYQTPLLIRSGITGLEVTYAADEKALFHEIANIIKRYDPDILLGYEIQMHSWGYLLQRAAALSIDLCRMISRVPDDKIENRFAAERDEYGSYTMSEINIVGRITLNLWRIMRNEVALTNYTFENVSFHVLHQRFPLFTFRVLSDWFDNKTDLYRWKMVDHYVSRVRGNLQMLEQLDLIGKTSEMARLFGIQFLHVLTRGSQYRVESMMLRIAKPMNYIPVTPSVQQRSQMRAPQCVPLIMEPESRFYSNSVLVLDFQSLYPSIVIAYNYCFSTCLGHVENLGKYDEFKFGCTSLRVPPDLLYQVRHDITVSPNGVAFVKPSVRKGVLPRMLEEILKTRFMVKQSMKAYKQDRALSRMLDARQLGLKLIANVTFGYTSANFSGRMPCIEVGDSIVHKARETLERAIKLVNDTKKWGARVVYGDTDSMFVLLKGATKEQSFKIGQEIAEAVTATNPKPVKLKFEKVYLPCVLQTKKRYVGYMYETLDQKDPVFDAKGIETVRRDSCPAVSKILERSLKLLFETRDISLIKQYVQRQCMKLLEGKASIQDFIFAKEYRGSFSYKPGACVPALELTRKMLTYDRRSEPQVGERVPYVIIYGTPGVPLIQLVRRPVEVLQDPTLRLNATYYITKQILPPLARIFSLIGIDVFSWYHELPRIHKATSSSRSEPEGRKGTISQYFTTLHCPVCDDLTQHGICSKCRSQPQHVAVILNQEIRELERQQEQLVKICKNCTGCFDRHIPCVSLNCPVLFKLSRVNRELSKAPYLRQLLDQF

SEQ ID NO: 3 - protein, 210 aa, Homo sapiens
MTTLTRQDLNFGQVVADVLCEFLEVAVHLILYVREVYPVGIFQKRKKYNVPVQMSCHPELNQYIQDTHCVKPLLEKNDVEKVVVVILDKEHRPVEKFVFEITQPPLLSISSDSLLSHVEQLLRAFILKISVCDAVLDHNPPGCTFTVLVHTREAATRNMEKIQVIKDFPWILADEQDVHMHDPRLIPLKTMTSDILKMQLYVEERAHKGS

SEQ ID NO: 4 - protein, 713 aa, Homo sapiens
MATGQDRVVALVDMDCFFVQVEQRQNPHLRNKPCAVVQYKSWKGGGIIAVSYEARAFGVTRSMWADDAKKLCPDLLLAQVRESRGKANLTKYREASVEVMEIMSRFAVIERASIDEAYVDLTSAVQERLQKLQGQPISADLLPSTYIEGLPQGPTTAEETVQKEGMRKQGLFQWLDSLQIDNLTSPDLQLTVGAVIVEEMRAAIERETGFQCSAGISHNKVLAKLACGLNKPNRQTLVSHGSVPQLFSQMPIRKIRSLGGKLGASVIEILGIEYMGELTQFTESQLQSHFGEKNGSWLYAMCRGIEHDPVKPRQLPKTIGCSKNFPGKTALATREQVQWWLLQLAQELEERLTKDRNDNDRVATQLVVSIRVQGDKRLSSLRRCCALTRYDAHKMSHDAFTVIKNCNTSGIQTEWSPPLTMLFLCATKFSASAPSSSTDITSFLSSDPSSLPKVPVTSSEAKTQGSGPAVTATKKATTSLESFFQKAAERQKVKEASLSSLTAPTQAPMSNSPSKPSLPFQTSQSTGTEPFFKQKSLLLKQKQLNNSSVSSPQQNPWSNCKALPNSLPTEYPGCVPVCEGVSKLEESSKATPAEMDLAHNSQSMHASSASKSVLEVTQKATPNPSLLAAEDQVPCEKCGSLVPVWDMPEHMDYHFALELQKSFLQPHSSNPQVVSAVSHQGKRNPKSPLACTNKRPRPEGMQTLESFFKPLTH

SEQ ID NO: 5 - protein, 870 aa, Homo sapiens
MDSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGIAPNTMLAKVCSDKNKPNGQYQILPNRQAVMDFIKDLPIRKVSGIGKVTEKMLKALGIITCTELYQQRALLSLLFSETSWHYFLHISLGLGSTHLTRDGERKSMSVERTFSEINKAEEQYSLCQELCSELAQDLQKERLKGRTVTIKLKNVNFEVKTRASTVSSVVSTAEEIFAIAKELLKTEIDADFPHPLRLRLMGVRISSFPNEEDRKHQQRSIIGFLQAGNQALSATECTLEKTDKDKFVKPLEMSHKKSFFDKKRSERKWSHQDTFKCEAVNKQSFQTSQPFQVLKKKMNENLEISENSDDCQILTCPVCFRAQGCISLEALNKHVDECLDGPSISENFKMFSCSHVSATKVNKKENVPASSLCEKQDYEAHPKIKEISSVDCIALVDTIDNSSKAESIDALSNKHSKEECSSLPSKSFNIEHCHQNSSSTVSLENEDVGSFRQEYRQPYLCEVKTGQALVCPVCNVEQKTSDLTLFNVHVDVCLNKSFIQELRKDKFNPVNQPKESSRSTGSSSGVQKAVTRTKRPGLMTKYSTSKKIKPNNPKHTLDIFFK

SEQ ID NO: 6 - protein, 740 aa, Homo sapiens
MEKLGVEPEEEGGGDDDEEDAEAWAMELADVGAAASSQGVHDQVLPTPNASSRVIVHVDLDCFYAQVEMISNPELKDKPLGVQQKYLVVTCNYEARKLGVKKLMNVRDAKEKCPQLVLVNGEDLTRYREMSYKVTELLEEFSPVVERLGFDENFVDLTEMVEKRLQQLQSDELSAVTVSGHVYNNQSINLLDVLHIRLLVGSQIAAEMREAMYNQLGLTGCAGVASNKLLAKLVSGVFKPNQQTVLLPESCQHLIHSLNHIKEIPGIGYKTAKCLEALGINSVRDLQTFSPKILEKELGISVAQRIQKLSFGEDNSPVILSGPPQSFSEEDSFKKCSSEVEAKNKIEELLASLLNRVCQDGRKPHTVRLIIRRYSSEKHYGRESRQCPIPSHVIQKLGTGNYDVMTPMVDILMKLFRNMVNVKMPFHLTLLSVCFCNLKALNTAKKGLIDYYLMPSLSTTSRSGKHSFKMKDTHMEDFPKDKETNRDFLPSGRIESTRTRESPLDTTNFSKEKDINEFPLCSLPEGVDQEVFKQLPVDIQEEILSGKSREKFQGKGSVSCPLHASRGVLSFFSKKQMQDIPINPRDHLSSSKQVSSVSPCEPGTSGFNSSSSSYMSSQKDYSYYLDNRLKDERISQGPKEPQGFHFTNSNPAVSAFHSFPNLQSEQLFSRNHTTDSHKQTVATDSHEGLTENREPDSVDEKITFPSDIDPQVFYELPEAVQKELLAEWKRAGSDFHIGHK

SEQ ID NO: 7 - protein, 1251 aa, Homo sapiens
MRRGGWRKRAENDGWETWGGYMAAKVQKLEEQFRSDAAMQKDGTSSTIFSGVAIYVNGYTDPSAEELRKLMMLHGGQYHVYYSRSKTTHIIATNLPNAKIKELKGEKVIRPEWIVESIKAGRLLSYIPYQLYTKQSSVQKGLSFNPVCRPEDPLPGPSNIAKQLNNRVNHIVKKIETENEVKVNGMNSWNEEDENNDFSFVDLEQTSPGRKQNGIPHPRGSTAIFNGHTPSSNGALKTQDCLVPMVNSVASRLSPAFSQEEDKAEKSSTDFRDCTLQQLQQSTRNTDALRNPHRTNSFSLSPLHSNTKINGAHHSTVQGPSSTKSTSSVSTFSKAAPSVPSKPSDCNFISNFYSHSRLHHISMWKCELTEFVNTLQRQSNGIFPGREKLKKMKTGRSALVVTDTGDMSVLNSPRHQSCIMHVDMDCFFVSVGIRNRPDLKGKPVAVTSNRGTGRAPLRPGANPQLEWQYYQNKILKGKAADIPDSSLWENPDSAQANGIDSVLSRAEIASCSYEARQLGIKNGMFFGHAKQLCPNLQAVPYDFHAYKEVAQTLYETLASYTHNIEAVSCDEALVDITEILAETKLTPDEFANAVRMEIKDQTKCAASVGIGSNILLARMATRKAKPDGQYHLKPEEVDDFIRGQLVTNLPGVGHSMESKLASLGIKTCGDLQYMTMAKLQKEFGPKTGQMLYRFCRGLDDRPVRTEKERKSVSAEINYGIRFTQPKEAEAFLLSLSEEIQRRLEATGMKGKRLTLKIMVRKPGAPVETAKFGGHGICDNIARTVTLDQATDNAKIIGKAMLNMFHTMKLNISDMRGVGIHVNQLVPTNLNPSTCPSRPSVQSSHFPSGSYSVRDVFQVQKAKKSTEEEHKEVFRAAVDLEISSASRTCTFLPPFPAHLPTSPDTNKAESSGKWNGLHTPVSVQSRLNLSIEVPSPSQLDQSVLEALPPDLREQVEQVCAVQQAESHGDKKKEPVNGCNTGILPQPVGTVLLQIPEPQESNSDAGINLIALPAFSQVDPEVFAALPAELQRELKAAYDQRQRQGENSTHQQSASASVPKNPLLHLKAAVKEKKRNKKKKTIGSPKRIQSPLNNKLLNSPAKTLPGACGSPQKLIDGFLKHEGPPAEKPLEELSASTSGVPGLSSLQSDPAGCVRPPAPNLAGAVEFNDVKTLLREWITTISDPMEEDILQVVKYCTDLIEEKDLEKLDLVIKYMKRLMQQSVESVWNMAFDFILDNVQVVLQQTYGSTLKVT

SEQ ID NO: 8 - protein, 2590 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 9 - protein, 2644 aa, Homo sapiens
MGEHGLELASMIPALRELGSATPEEYNTVVQKPRQILCQFIDRILTDVNVVAVELVKKTDSQPTSVMLLDFIQHIMKSSPLMFVNVSGSHEAKGSCIEFSNWIITRLLRIAATPSCHLLHKKICEVICSLLFLFKSKSPAIFGVLTKELLQLFEDLVYLHRRNVMGHAVEWPVVMSRFLSQLDEHMGYLQSAPLQLMSMQNLEFIEVTLLMVLTRIIAIVFFRRQELLLWQIGCVLLEYGSPKIKSLAISFLTELFQLGGLPAQPASTFFSSFLELLKHLVEMDTDQLKLYEEPLSKLIKTLFPFEAEAYRNIEPVYLNMLLEKLCVMFEDGVLMRLKSDLLKAALCHLLQYFLKFVPAGYESALQVRKVYVRNICKALLDVLGIEVDAEYLLGPLYAALKMESMEIIEEIQCQTQQENLSSNSDGISPKRRRLSSSLNPSKRAPKQTEEIKHVDMNQKSILWSALKQKAESLQISLEYSGLKNPVIEMLEGIAVVLQLTALCTVHCSHQNMNCRTFKDCQHKSKKKPSVVITWMSLDFYTKVLKSCRSLLESVQKLDLEATIDKVVKIYDALIYMQVNSSFEDHILEDLCGMLSLPWIYSHSDDGCLKLTTFAANLLTLSCRISDSYSPQAQSRCVFLLTLFPRRIFLEWRTAVYNWALQSSHEVIRASCVSGFFILLQQQNSCNRVPKILIDKVKDDSDIVKKEFASILGQLVCTLHGMFYLTSSLTEPFSEHGHVDLFCRNLKATSQHECSSSQLKASVCKPFLFLLKKKIPSPVKLAFIDNLHHLCKHLDFREDETDVKAVLGTLLNLMEDPDKDVRVAFSGNIKHILESLDSEDGFIKELFVLRMKEAYTHAQISRNNELKDTLILTTGDIGRAAKGDLVPFALLHLLHCLLSKSASVSGAAYTEIRALVAAKSVKLQSFFSQYKKPICQFLVESLHSSQMTALPNTPCQNADVRKQDVAHQREMALNTLSEIANVFDFPDLNRFLTRTLQVLLPDLAAKASPAASALIRTLGKQLNVNRREILINNFKYIFSHLVCSCSKDELERALHYLKNETEIELGSLLRQDFQGLHNELLLRIGEHYQQVFNGLSILASFASSDDPYQGPRDIISPELMADYLQPKLLGILAFFNMQLLSSSVGIEDKKMALNSLMSLMKLMGPKHVSSVRVKMMTTLRTGLRFKDDFPELCCRAWDCFVRCLDHACLGSLLSHVIVALLPLIHIQPKETAAIFHYLIIENRDAVQDFLHEIYFLPDHPELKKIKAVLQEYRKETSESTDLQTTLQLSMKAIQHENVDVRIHALTSLKETLYKNQEKLIKYATDSETVEPIISQLVTVLLKGCQDANSQARLLCGECLGELGAIDPGRLDFSTTETQGKDFTFVTGVEDSSFAYGLLMELTRAYLAYADNSRAQDSAAYAIQELLSIYDCREMETNGPGHQLWRRFPEHVREILEPHLNTRYKSSQKSTDWSGVKKPIYLSKLGSNFAEWSASWAGYLITKVRHDLASKIFTCCSIMMKHDFKVTIYLLPHILVYVLLGCNQEDQQEVYAEIMAVLKHDDQHTINTQDIASDLCQLSTQTVFSMLDHLTQWARHKFQALKAEKCPHSKSNRNKVDSMVSTVDYEDYQSVTRFLDLIPQDTLAVASFRSKAYTRAVMHFESFITEKKQNIQEHLGFLQKLYAAMHEPDGVAGVSAIRKAEPSLKEQILEHESLGLLRDATACYDRAIQLEPDQIIHYHGVVKSMLGLGQLSTVITQVNGVHANRSEWTDELNTYRVEAAWKLSQWDLVENYLAADGKSTTWSVRLGQLLLSAKKRDITAFYDSLKLVRAEQIVPLSAASFERGSYQRGYEYIVRLHMLCELEHSIKPLFQHSPGDSSQEDSLNWVARLEMTQNSYRAKEPILALRRALLSLNKRPDYNEMVGECWLQSARVARKAGHHQTAYNALLNAGESRLAELYVERAKWLWSKGDVHQALIVLQKGVELCFPENETPPEGKNMLIHGRAMLLVGRFMEETANFESNAIMKKYKDVTACLPEWEDGHFYLAKYYDKLMPMVTDNKMEKQGDLIRYIVLHFGRSLQYGNQFIYQSMPRMLTLWLDYGTKAYEWEKAGRSDRVQMRNDLGKINKVITEHTNYLAPYQFLTAFSQLISRICHSHDEVFVVLMEIIAKVFLAYPQQAMWMMTAVSKSSYPMRVNRCKEILNKAIHMKKSLEKFVGDATRLTDKLLELCNKPVDGSSSTLSMSTHFKMLKKLVEEATFSEILIPLQSVMIPTLPSILGTHANHASHEPFPGHWAYIAGFDDMVEILASLQKPKKISLKGSDGKFYIMMCKPKDDLRKDCRLMEFNSLINKCLRKDAESRRRELHIRTYAVIPLNDECGIIEWVNNTAGLRPILTKLYKEKGVYMTGKELRQCMLPKSAALSEKLKVFREFLLPRHPPIFHEWFLRTFPDPTSWYSSRSAYCRSTAVMSMVGYILGLGDRHGENILFDSLTGECVHVDFNCLFNKGETFEVPEIVPFRLTHNMVNGMGPMGTEGLFRRACEVTMRLMRDQREPLMSVLKTFLHDPLVEWSKPVKGHSKAPLNETGEVVNEKAKTHVLDIEQRLQGVIKTRNRVTGLPLSIEGHVHYLIQEATDENLLCQMYLGWTPYM

SEQ ID NO: 10 - protein, 476 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQVTISTTDRRNNKLIFKVNLLEMDDKILVDFRLSKGDGLEFKRHFLKIKGKLIDIVSSQKIWLPAT

SEQ ID NO: 11 - protein, 608 aa, Homo sapiens
MSQEGDYGRWTISSSDESEEEKPKPDKPSTSSLLCARQGAANEPRYTCSEAQKAAHKRKISPVKFSNTDSVLPPKRQKSGSQEDLGWCLSSSDDELQPEMPQKQAEKVVIKKEKDISAPNDGTAQRTENHGAPACHRLKEEEDEYETSGEGQDIWDMLDKGNPFQFYLTRVSGVKPKYNSGALHIKDILSPLFGTLVSSAQFNYCFDVDWLVKQYPPEFRKKPILLVHGDKREAKAHLHAQAKPYENISLCQAKLDIAFGTHHTKMMLLLYEEGLRVVIHTSNLIHADWHQKTQGIWLSPLYPRIADGTHKSGESPTHFKADLISYLMAYNAPSLKEWIDVIHKHDLSETNVYLIGSTPGRFQGSQKDNWGHFRLKKLLKDHASSMPNAESWPVVGQFSSVGSLGADESKWLCSEFKESMLTLGKESKTPGKSSVPLYLIYPSVENVRTSLEGYPAGGSLPYSIQTAEKQNWLHSYFHKWSAETSGRSNAMPHIKTYMRPSPDFSKIAWFLVTSANLSKAAWGALEKNGTQLMIRSYELGVLFLPSAFGLDSFKVKQKFFAGSQEPMATFPVPYDLPPELYGSKDRPWIWNIPYVKAPDTHGNMWVPS

SEQ ID NO: 12 - protein, 1249 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTSPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 13 - protein, 633 aa, Homo sapiens
MPEIRLRHVVSCSSQDSTHCAENLLKADTYRKWRAAKAGEKTISVVLQLEKEEQIHSVDIGNDGSAFVEVLVGSSAGGAGEQDYEVLLVTSSFMSPSESRSGSNPNRVRMFGPDKLVRAAAEKRWDRVKIVCSQPYSKDSPFGLSFVRFHSPPDKDEAEAPSQKVTVTKLGQFRVKEEDESANSLRPGALFFSRINKTSPVTASDPAGPSYAAATLQASSAASSASPVSRAIGSTSKPQESPKGKRKLDLNQEEKKTPSKPPAQLSPSVPKRPKLPAPTRTPATAPVPARAQGAVTGKPRGEGTEPRRPRAGPEELGKILQGVVVVLSGFQNPFRSELRDKALELGAKYRPDWTRDSTHLICAFANTPKYSQVLGLGGRIVRKEWVLDCHRMRRRLPSQRYLMAGPGSSSEEDEASHSGGSGDEAPKLPQKQPQTKTKPTQAAGPSSPQKPPTPEETKAASPVLQEDIDIEGVQSEGQDNGAEDSGDTEDELRRVAEQKEHRLPPGQEENGEDPYAGSTDENTDSEEHQEPPDLPVPELPDFFQGKHFFLYGEFPGDERRKLIRYVTAFNGELEDYMSDRVQFVITAQEWDPSFEEALMDNPSLAFVRPRWIYSCNEKQKLLPHQLYGVVPQA

(19) United States
(12) Patent Application Publication
(10) Pub. No.: US 2025/0263799 A1
(43) Pub. Date: Aug. 21, 2025

(54) METHODS OF TREATING CANCER USING REPLICATION STRESS MODULATORS

(71) Applicants: The Institute of Cancer Research: Royal Cancer Hospital, London (GB); Breast Cancer Now, London (GB); King's College London, London (GB)

(72) Inventors: Daniel Beckford Weekes, London (GB); Dalia Tarantino, London (GB); Christopher James Lord, London (GB); Stephen John Pettitt, London (GB); Andrew Nicholas James Tutt, London (GB); Callum Reece Walker, London (GB); Anita Grigoriadis, London (GB)

(21) Appl. No.: 18/858,434

(22) PCT Filed: Apr. 21, 2023
(86) PCT No.: PCT/EP2023/060509

§ 371 Date: Oct. 21, 2024

(30) Foreign Application Priority Data
Apr. 21, 2022 (LU) LU102932

(51) Int. Cl. C12Q 1/6886; A61K 31/37; A61K 31/4439

(52) U.S. Cl. CPC C12Q 1/6886; A61K 31/37; A61K 31/4439; C12Q 2600/106; C12Q 2600/158

(57) ABSTRACT

An agent that generates DNA replication stress and/or inhibits pathways involved in DNA replication stress tolerance for use in a method of treating a patient with cancer. The treatment comprises: determining whether the cancer expresses HORMAD1; and, if so, administering to said patient an agent that generates DNA replication stress and/or inhibits pathways involved in DNA replication stress tolerance.

### FIELD OF THE INVENTION

The present invention relates to the treatment of cancer and, in particular, to the treatment of patients whose cancer expresses HORMAD1, with an agent that generates DNA replication stress and/or inhibits pathways involved in DNA replication stress tolerance; and diagnostic methods thereof.

### BACKGROUND OF THE INVENTION

Cancer is a condition in which cells in a part of the body experience out-of-control growth, and is one of the most life threatening diseases. For decades surgery, chemotherapy, and radiation were the established treatments for various cancers. Patients usually receive a combination of these treatments depending upon the type and extent of their disease. But chemotherapy is the most important option for cancer patients when surgical treatment (i.e. the successful removal of all diseased tissue) is not possible. While surgery is sometimes effective in removing tumours located at certain sites, for example, in the breast, colon, and skin, it cannot be used in the treatment of tumours located in other areas, such as the backbone, nor in the treatment of disseminated hematological cancers including cancers of the blood and blood-forming tissues (such as the bone marrow). In addition, chemotherapy is often used to supplement surgery to treat any diseased tissue that remained in the patient following surgery. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. One of the main causes of failure in chemotherapy is the development of drug resistance by the cancer cells, a serious problem that may lead to recurrence of disease or even death. Chemotherapy can also cause side effects as the drugs can also affect normal healthy cells as well as cancerous cells. For these and other reasons there remains a need for more effective chemotherapeutic options for treating cancers.

One area of exploration is personalised or precision medicine, where treatments can be selected based on a genetic understanding of the disease. Personalised medicine is a form of medicine that uses information about a person's own genes or proteins to inform a treatment strategy. In cancer, personalized medicine uses specific information about a person's tumor to help make a diagnosis, plan treatment, find out how well treatment is working, or make a prognosis. Examples of personalized medicine include using targeted therapies to treat specific types of cancer cells, such as HER2-positive breast cancer cells, or using tumor marker testing to help diagnose cancer.

Through the identification of new-targeted cancer therapies it may be possible to develop treatments which are more efficacious and/or exhibit reduced side effects in comparison to traditional chemotherapy options.

### SUMMARY OF THE INVENTION

According to an aspect of the present invention, there is provided an agent that generates DNA replication stress and/or inhibits pathways involved in DNA replication stress tolerance for use in a method of treating a patient with cancer, said treatment comprising:
a) determining whether the cancer expresses HORMAD1; and, if so
b) administering to said patient an agent that generates DNA replication stress and/or inhibits pathways involved in DNA replication stress tolerance.

In a further aspect, there is provided an agent that generates DNA replication stress and/or inhibits pathways involved in DNA replication stress tolerance for use in a method of treating a patient with cancer, said treatment comprising:
a) determining whether a test sample from the patient expresses HORMAD1; and, if so
b) administering to said patient an agent that generates DNA replication stress and/or inhibits pathways involved in DNA replication stress tolerance.

In a further aspect, there is provided a composition comprising an agent that generates DNA replication stress and/or inhibits pathways involved in DNA replication stress tolerance for use in a method of treating a patient with cancer, said treatment comprising:
a) determining whether the cancer expresses HORMAD1; and, if so
b) administering to said patient a composition comprising an agent that generates DNA replication stress and/or inhibits pathways involved in DNA replication stress tolerance.

In a further aspect, there is provided a composition comprising an agent that generates DNA replication stress and/or inhibits pathways involved in DNA replication stress tolerance for use in a method of treating a patient with cancer, said treatment comprising:
a) determining whether a test sample from the patient expresses HORMAD1; and, if so
b) administering to said patient a composition comprising an agent that generates DNA replication stress and/or inhibits pathways involved in DNA replication stress tolerance.

In an embodiment, the agent may modulate the expression and/or activity of DNA polymerase theta.

In an embodiment, the agent may be a DNA polymerase theta inhibitor.

In an embodiment, the DNA polymerase theta inhibitor may be ART558, or a pharmaceutically acceptable salt thereof.

In an embodiment, the DNA polymerase inhibitor may be Novobiocin, or a pharmaceutically acceptable salt thereof.

In an embodiment, the agent may modulate the expression and/or activity of one or more translesion synthesis (TLS) polymerases.

In an embodiment, the one or more TLS polymerases may be selected from REV1, POLH, POLK, and POLζ.

In an embodiment, POLζ may comprise REV3L and REV7 subunits, and modulating the expression and/or activity of POLζ may comprise modulating the activity and/or expression of one or both of REV3L and REV7.

In an embodiment, the agent may modulate the expression and/or activity of one or more cell-cycle checkpoint kinases, optionally ATR and/or CHK1.

In an embodiment, the agent or composition may modulate the expression and/or activity of TDP1.

In an embodiment, the agent may modulate the expression and/or activity of BRIP1.

In an embodiment, the agent or composition may modulate the expression and/or activity of XRCC1.

In an embodiment, the cancer may be a HORMAD1 positive cancer selected from breast cancers, such as triple negative (ER, PgR, and HER2 negative) and/or basal like breast cancers, leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.

In a further aspect, there is provided an agent that generates DNA replication stress and/or inhibits pathways involved in DNA replication stress tolerance, for use in the treatment of a HORMAD1 positive cancer.

In a further aspect, there is provided a composition comprising an agent that generates DNA replication stress and/or inhibits pathways involved in DNA replication stress for use in the treatment of a HORMAD1 positive cancer.

In an embodiment, the cancer may be selected from breast cancers, such as triple negative (ER, PgR, and HER2 negative) and/or basal like breast cancers, leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.

In a further aspect, there is provided a method of treating a patient with cancer, said method comprising:
a) determining whether said cancer expresses HORMAD1; and, if so
b) administering to said patient an agent that generates DNA replication stress and/or inhibits pathways involved in DNA replication stress tolerance or a composition comprising said agent.

In a further aspect, there is provided a method of treating a patient with cancer, said method comprising:
a) determining whether a test sample from said patient expresses HORMAD1; and, if so
b) administering to said patient an agent that generates DNA replication stress and/or inhibits pathways involved in DNA replication stress tolerance or a composition comprising said agent.

In a further aspect, there is provided an in-vitro method for identifying an individual with cancer having suitability for treatment with an agent that generates DNA replication stress and/or inhibits pathways involved in DNA replication stress tolerance or a composition comprising said agent, said method comprising determining whether a cell sample from said individual expresses HORMAD1.

In a further aspect, there is provided an agent that modulates the expression and/or activity of DNA polymerase theta for use in a method of treating a patient with cancer, said treatment comprising:
a) determining whether the cancer, or a test sample from said patient, expresses HORMAD1; and, if so
b) administering to said patient an agent that modulates the expression and/or activity of DNA polymerase theta.

In a further aspect, there is provided an agent that modulates the expression and/or activity of one or more translesion synthesis (TLS) polymerases for use in a method of treating a patient with cancer, said treatment comprising:
a) determining whether the cancer, or a test sample from said patient, expresses HORMAD1; and, if so
b) administering to said patient an agent that modulates the expression and/or activity of one or more translesion synthesis (TLS) polymerases.

In a further aspect, there is provided an agent that modulates the expression and/or activity of one or more cell-cycle checkpoint kinases, optionally ATR and/or CHK1, for use in a method of treating a patient with cancer, said treatment comprising:
a) determining whether the cancer, or a test sample from said patient, expresses HORMAD1; and, if so
b) administering to said patient an agent that modulates the expression and/or activity of one or more cell-cycle checkpoint kinases, optionally ATR and/or CHK1.

In a further aspect, there is provided an agent that modulates the expression and/or activity of TDP1 for use in a method of treating a patient with cancer, said treatment comprising:
a) determining whether the cancer, or a test sample from said patient, expresses HORMAD1; and, if so
b) administering to said patient an agent that modulates the expression and/or activity of TDP1.

In a further aspect, there is provided an agent that modulates the expression and/or activity of BRIP1 for use in a method of treating a patient with cancer, said treatment comprising:
a) determining whether the cancer, or a test sample from said patient, expresses HORMAD1; and, if so
b) administering to said patient an agent that modulates the expression and/or activity of BRIP1.

In a further aspect, there is provided an agent that modulates the expression and/or activity of XRCC1 for use in a method of treating a patient with cancer, said treatment comprising:
a) determining whether the cancer, or a test sample from said patient, expresses HORMAD1; and, if so
b) administering to said patient an agent that modulates the expression and/or activity of XRCC1.

In a further aspect, there is provided an agent that modulates the expression and/or activity of DNA polymerase theta, one or more translesion synthesis (TLS) polymerases, one or more cell-cycle checkpoint kinases (optionally ATR and/or CHK1), TDP1, BRIP1 and/or XRCC1, for use in the treatment of a HORMAD1 positive cancer.

In a further aspect, there is provided a composition comprising an agent that modulates the expression and/or activity of DNA polymerase theta, one or more translesion synthesis (TLS) polymerases, one or more cell-cycle checkpoint kinases (optionally ATR and/or CHK1), TDP1, BRIP1 and/or XRCC1, for use in the treatment of a HORMAD1 positive cancer. In a further aspect, there is provided a method of treating a patient with cancer, said method comprising:
a) determining whether said cancer, or a test sample from said patient, expresses HORMAD1; and, if so
b) administering to said patient an agent that modulates the expression and/or activity of DNA polymerase theta, one or more translesion synthesis (TLS) polymerases, one or more cell-cycle checkpoint kinases (optionally ATR and/or CHK1), TDP1, BRIP1 and/or XRCC1, or a composition comprising said agent.

In a further aspect, there is provided an in-vitro method for identifying an individual with cancer having suitability for treatment with an agent that modulates the expression and/or activity of DNA polymerase theta, one or more translesion synthesis (TLS) polymerases, one or more cell-cycle checkpoint kinases (optionally ATR and/or CHK1), TDP1, BRIP1 and/or XRCC1, or a composition comprising said agent, said method comprising determining whether a cell sample from said individual expresses HORMAD1.

### DESCRIPTION OF THE FIGURES

FIGS. 1, A-C shows HORMAD1 gene expression in multiple cancers but not in normal somatic tissues.

FIG. 2, A shows levels of HORMAD1 expression achieved in two clones (H1-clone 1 and H1-clone 2) of SUM159 cells engineered to express HORMAD1 when exposed to doxycycline. B shows the level of HORMAD1 expression in H1-clone 1 cells in comparison to that found in the endogenous HORMAD1 expressing breast cancer line mdamb436. C-D show the proportion of nuclei with >5 γH2AX foci in HORMAD1 expressing SUM159 cells. E-H shows the number of aberrant nuclear structures in HORMAD1 expressing SUM159 cells compared to control SUM159 cells engineered to express GFP upon doxycycline induction.

FIG. 3 shows RNAi screen quality control data. A-B Representative Z′ factor analysis showing the distribution of positive (siPLK1 (lower plots in A, and left-hand curve in B)) and negative control (siCON1, siCON2, ALLSTAR (upper plots in A, and right-hand curve in B)) Z-score values. A Z′ factor value >0.3 indicates a good separation of positive and negative control Z-score values. C Boxplot illustrating the distribution of Z-scores of positive and negative control siRNAs for each technical replicate in the clonally-derived HORMAD1-inducible SUM159 doxycycline experimental arm. Boxes represent individual Z-scores and error bars represent the SD. D Representative Spearman's correlation analysis of Z-scores from replicate 1 and 2 in the clonally-derived HORMAD1-inducible SUM159 doxycycline experimental arm (r²=0.96).

FIG. 4 shows bar plots displaying increased normalised percentage inhibition (NPI) of clonally-derived HORMAD1-inducible SUM159 cells (+DOX/+HORMAD1 (right-hand bars) vs. −DOX/−HORMAD1 (left-hand bars)) transfected with an siRNA pool or four individual siRNAs targeting ATR (A), BRIP1 (B), POLH (C), TDP1 (D) and XRCC1 (E) and exposed to HORMAD1 expression for 4 days. Non-targeting (siALLSTAR) and targeting (siPLK1) siRNAs were used as normalisation controls. Error bars indicate SD from mean effects (n=3), p values represent multiple Student t tests (p=***<0.0001, p=**<0.001, p=*<0.05).

FIG. 5 shows bars plots displaying increased normalised percentage inhibition (NPI) of clonally-derived GFP-inducible SUM159 cells (+DOX/+GFP (right-hand bars) vs. −DOX/−GFP (left-hand bars)) (A, C, E, G and I) and SUM159 parental cells (B, D, and F, H and J) transfected with an siRNA pool or four individual siRNAs targeting ATR (A, B), BRIP1 (C, D), POLH (E, F), TDP1 (G, H) and XRCC1 (I, J). Non-targeting (siALLSTAR) and targeting (siPLK1) siRNAs were used as normalisation controls. Error bars indicate SD from mean effects (n=3), p values represent multiple Student t tests (p=***<0.0001, p=**<0.001, p=*<0.05).

FIG. 6 shows bar plots displaying the reduction in gene expression for each of ATR (A), BRIP1 (B), POLH (C), TDP1 (D) and XRCC1 (E) in response to each siRNA oligonucleotide and siRNA pool, as measured by RT-qPCR analysis (right-hand bars=+HORMAD, left-hand bars=−HORMAD).

FIG. 7 shows expression levels of HORMAD1 in isogenic doxycycline-inducible HA tagged-HORMAD1 expressing models of the non-transformed cell lines MCF10A (A) and RPE1 (B). E and F compare the expression levels of HORMAD1 in the HORMAD1 expressing MCF10A (E) and RPE1 (F) models, to that found in the endogenous HORMAD1 expressing breast cancer line MDAMB436. C and D compare cellular growth in MCF10A (C) and RPE1 (D) cells with and without HORMAD1 expression, as measured by time-lapsed microscopy (lower plot=+HORMAD, upper plot=−HORMAD).

FIG. 8 shows that HORMAD1 drives ATR, BRIP1, POLH, TDP1 and XRCC1 dependencies in multiple cellular models. A Bar plot displaying reduced colony counts of MCF10A cells (+DOX/+HORMAD1 vs. −DOX/−HORMAD1) transfected with an siRNA pool targeting ATR, BRIP1, POLH, TDP1 and XRCC1 exposed to HORMAD1 expression for 14 days (in total). Non-targeting (siALLSTAR) siRNA was used as normalisation control. Error bars indicate SD from mean effects (n=3), p values represent multiple Student t tests. B Representative colony images from experiment A. C Bar plot displaying the percentage of ATR, BRIP1, POLH, TDP1 and XRCC1 mRNA expression following siRNA-mediated gene knockdown for experiments described in A, measured by RT-qPCR and normalised to ACTB (right-hand bars=+HORMAD, left-hand bars=−HORMAD). D Bar plot displaying reduced colony counts of RPE1 cells (+DOX/+HORMAD1 vs. −DOX/−HORMAD1) transfected with an siRNA pool targeting ATR, BRIP1, POLH, TDP1 and XRCC1 and exposed to HORMAD1 expression for 14 days (in total). Non-targeting (siALLSTAR) siRNA was used as normalisation control. Error bars indicate SD from mean effects (n=3), p values represent multiple Student t tests. E Representative colony images from experiment D. F Bar plot displaying the percentage of ATR, BRIP1, POLH, TDP1 and XRCC1 mRNA expression following siRNA-mediated gene knockdown for experiments described in D, measured by RT-qPCR and normalised to ACTB (right-hand bars=+HORMAD, left-hand bars=−HORMAD).

FIG. 9 shows bar plots displaying normalised percentage inhibition (NPI) observed in siRNA mediated knockdown experiments in the HORMAD1 positive cell lines HCC38, BT549, HCC1143 and MDAMB436 for ATR (A), BRIP1 (B), TDP1 (C), POLH (D) and XRCC1 (E).

FIG. 10 compares the median Z scores between HORMAD1-negative (n=15) and HORMAD1-positive (n=8) breast cancer cell lines following RNAi mediated knockdown of ATR (A) and XRCC1 (C), and CRISPR-Cas9 mediated knockout of REV7 (also known as MAD2L2) (B). Depmap data was downloaded and overlaid with internal transcriptomic data for HORMAD1 expression. p values represent unpaired t tests.

FIG. 11 provides further validation of HORMAD1-driven POLH dependency. A Bar plot displaying reduced surviving fractions of clonally-derived HORMAD1-inducible SUM159 cells (+DOX/+HORMAD1 (right-hand bars) vs. −DOX/−HORMAD1 (left-hand bars)) transfected with an siRNA pool or 4 individual siRNAs targeting POLH and exposed to HORMAD1 expression for 14 days (in total). Non-targeting (siALLSTAR) and targeting (siPLK1) siRNAs were used as transfection controls and surviving fractions calculated from mock-transfected cells. Error bars indicate SD from mean effects (n=3), p values represent multiple Student t tests (p=***<0.0001, p=**<0.001, p=*<0.05). B Bar plot displaying the percentage of POLH mRNA expression following siRNA-mediated depletion of POLH described in A, measured by RT-qPCR and normalised to ACTB (right-hand bars=+DOX/+HORMAD1, left-hand bars=−DOX/−HORMAD1). C Bar plot displaying reduced surviving fractions of clonally-derived HORMAD1-inducible SUM159 cells (+DOX/+HORMAD1 (right-hand bars) vs. −DOX/−HORMAD1 (left-hand bars)) expressing constitutive Cas9-mCherry, transfected with 5 Edit-R crRNAs targeting POLH, and exposed to HORMAD1 expression for 14 days (in total). Surviving fractions were calculated relative to Cas9-expressing mock-transfected controls. Error bars indicate SD from mean effects (n=3), p values represent multiple Student t tests (p=***<0.0001, p=**<0.001, p=*<0.05). D Western blot analysis of POLH protein knockout from experiment C. E-F Left, growth curves displaying reduced cellular growth of HORMAD1-expressing breast cancer cell lines E HCC38 and F BT549 expressing constitutive Cas9-mCherry and bulk-transfected with 3 POLH-targeting Edit-R crRNAs (lower three plots=POLH-targeting Edit-R crRNAs, upper plot=mock). Cell number was normalised relative to T0 counts. Error bars indicate SD from mean effects (n=3). p values represent two-way repeated measures ANOVA. Right, western blot analysis of HORMAD1 expression and POLH protein knockout from experiments described in left panel.

FIG. 12 shows a significant decrease in cellular viability following continuous HORMAD1 expression for 21.5 weeks and siRNA-mediated depletion of POLH (A) confirmed by RT-qPCR (B) (right-hand bars=+HORMAD, left-hand bars=−HORMAD).

FIG. 13 shows that HORMAD1 drives broad genetic dependency on TLS polymerases. A Bar plot displaying reduced colony counts of SUM159 cells (+DOX/+HORMAD1 vs. −DOX/−HORMAD1) transfected with an siRNA pool targeting REV3L and REV7 and exposed to HORMAD1 expression for 14 days (in total). Non-targeting (siALLSTAR) siRNA was used as normalisation control. Error bars indicate SD from mean effects (n=3), p values represent multiple Student t tests. B Representative colony images from experiment A. C Bar plot displaying the percentage of REV3L and REV7 mRNA expression following siRNA-mediated gene knockdown for experiments described in A, measured by RT-qPCR and normalised to ACTB (right-hand bars=+HORMAD, left-hand bars=−HORMAD). D Bar plot displaying reduced colony counts of MCF10A cells (+DOX/+HORMAD1 vs. −DOX/−HORMAD1) transfected with an siRNA SMARTpool targeting POLK, REV1 and REV7 and exposed to HORMAD1 expression for 14 days (in total). Non-targeting (siALLSTAR) siRNA was used as normalisation control. Error bars indicate SD from mean effects (n=3), p values represent multiple Student t tests. E Representative colony images from experiment D. F Bar plot displaying the percentage of POLK, REV1 and REV7 mRNA expression following siRNA-mediated gene knockdown for experiments described in D, measured by RT-qPCR and normalised to ACTB (right-hand bars=+HORMAD, left-hand bars=−HORMAD). G Bar plot displaying reduced colony counts of RPE1 cells (+DOX/+HORMAD1 vs. −DOX/−HORMAD1) transfected with an siRNA SMARTpool targeting POLK, REV3L and REV7 and exposed to HORMAD1 expression for 14 days (in total). Non-targeting (siALLSTAR) siRNA was used as normalisation control. Error bars indicate SD from mean effects (n=3), p values represent multiple Student t tests. H Representative colony images from experiment G. I Bar plot displaying the percentage of POLK, REV3L and REV7 mRNA expression following siRNA-mediated gene knockdown for experiments described in G, measured by RT-qPCR and normalised to ACTB (right-hand bars=+HORMAD, left-hand bars=−HORMAD).

FIG. 14 shows normalised percentage inhibition (NPI) for siRNA mediated knockdown experiments in the HORMAD1 positive cell lines HCC38, BT549 HCC1143 and MDAMB436 for POLK (A), REV1 (B), REV3L (C) and REV7 (D).

FIG. 15 shows that expression of HORMAD1 increases the sensitivity of RPE-1 (A) and SUM159 (B) cells to siRNA mediated POLQ knockdown.

FIG. 16 shows that dox-induced expression of HORMAD1 increases sensitivity to the POLQ inhibitor ART558 in RPE1 (A) and SUM159 (B) cells, and to the POLQ inhibitor Novobiocin in RPE1 cells (C) (lower plots=+DOX, upper plots=−DOX).

FIGS. 17(A) and (B) show data corresponding to that of FIGS. 16(A) and (B) respectively, including further statistical analysis and data from an additional replicate in (A).

FIG. 18 shows that dox-induced expression of HORMAD1 increases sensitivity to the CHK1 inhibitors PF47736 (A), SAR020106 (B), and in SUM149 cells (lower plots=+DOX, upper plots=−DOX). C, Breast cancer cell lines were characterized as HORMAD1 negative and positive based on gene expression and the AUC of the CHK1 inhibitor LY2606368 plotted (Data obtained from depmap.org). D, SUM159 cells with dox-inducible expression of HORMAD1 were exposed to the indicated doses of the CHK1 inhibitor LY2606368 (lower plot=+DOX, upper plot=−DOX). Induction of HORMAD1 expression lead to increased sensitivity to LY2606368.

FIG. 19 shows that dox-inducible knock down of HORMAD1 in H1299 (A) and constitutive knock down of HORMAD1 in MDA-MB-436 (B) decreases sensitivity to POLQ inhibitors.

### DETAILED DESCRIPTION OF THE INVENTION

### Definitions

In the present application, a number of general terms and phrases are used, which should be interpreted as follows.

In one embodiment, “HORMAD1” may be alternatively known as CT46, NOHMA, or DKFZP434A1315. The Ensembl version number may be ENSG00000143452.16. It may refer to a nucleic acid sequence comprising a sequence defined in NCBI Reference Sequence: NM_001199829.2 or NM_032132.5. In one embodiment, HORMAD1 may comprise or consist of SEQ ID NO: 1.

The term “treating”, as used herein, unless otherwise indicated, means reversing, attenuating, alleviating or inhibiting the progress of the disease or condition to which such term applies, or one or more symptoms of such disorder or condition. The term “treatment”, as used herein, unless otherwise indicated, refers to the act of treating as “treating” is defined immediately above.

“Patient” includes humans, non-human mammals (e.g., dogs, cats, rabbits, cattle, horses, sheep, goats, swine, deer, and the like) and non-mammals (e.g., birds, and the like). Preferably, the patient is a human patient.

“Pharmaceutically acceptable salts” means salts of compounds of the present invention which are pharmaceutically acceptable, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacid salts.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The present invention has identified that expression of HORMAD1 in cancer sub-types can be exploited by agents that generate DNA replication stress and/or inhibit pathways involved in DNA replication stress tolerance to create targeted cancer therapies.

By “generate DNA replication stress” is meant to cause (either directly, or indirectly through effects on one or more biological pathways) DNA replication stress, for example (but not limited to) replication fork slowing, replication fork stalling, replication fork fastening and replication fork collapse. For example, the illegitimate activation of oncogenes such as CCNE1 (Cyclin E) cause deregulation of replication initiation, excessive replication origin firing and increased interference between replication and transcription, which together cause replication slowing and DNA damage (Jones et al., 2013).

By “pathways involved in DNA replication stress tolerance”, is meant pathways that prevent, resolve, or partially prevent or resolve DNA replication stress.

By “agent that inhibits pathways involved in DNA replication stress tolerance” is meant any agent that modulates the activity and/or expression of molecules (e.g. enzymes) involved in said pathways, for example those described herein. Preferably, the agent disrupts, impairs or inhibits the activity and/or expression of molecules required by, or which augment, said pathways. DNA replication stress can be quantified experimentally, for example by DNA fibre combing assays.

### HORMAD1

HORMAD1 is the mammalian homolog of HOP1, a meiotic HORMA-domain containing protein first identified in yeast. HORMAD1 regulates numerous aspects of meiotic cell behaviour, including chromosome homolog synapsis, the initiation and repair of SPO11-induced double-stranded DNA breaks, as well as the subsequent control over cell cycle checkpoints that permits the generation and maturation of gamete cells.

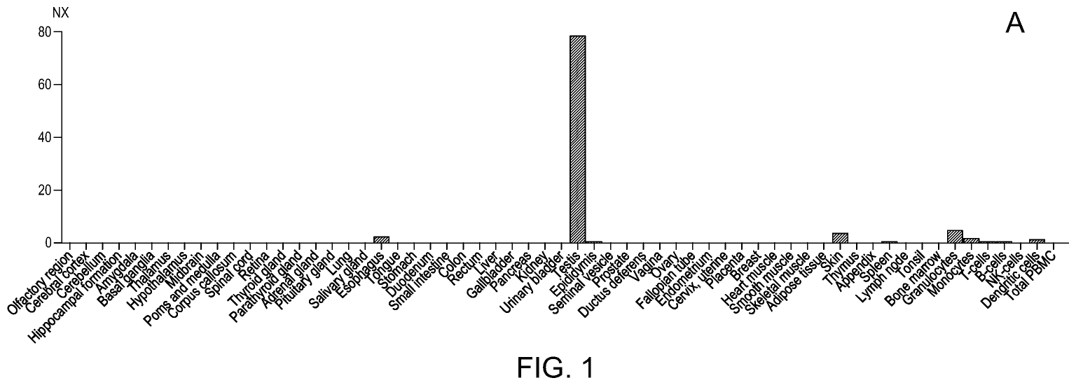

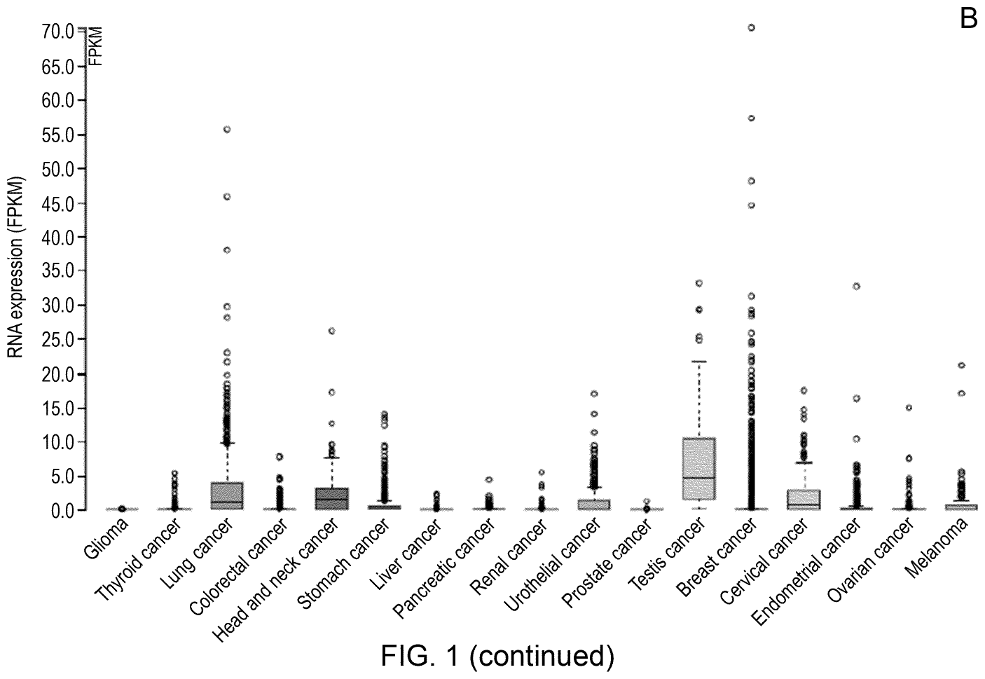

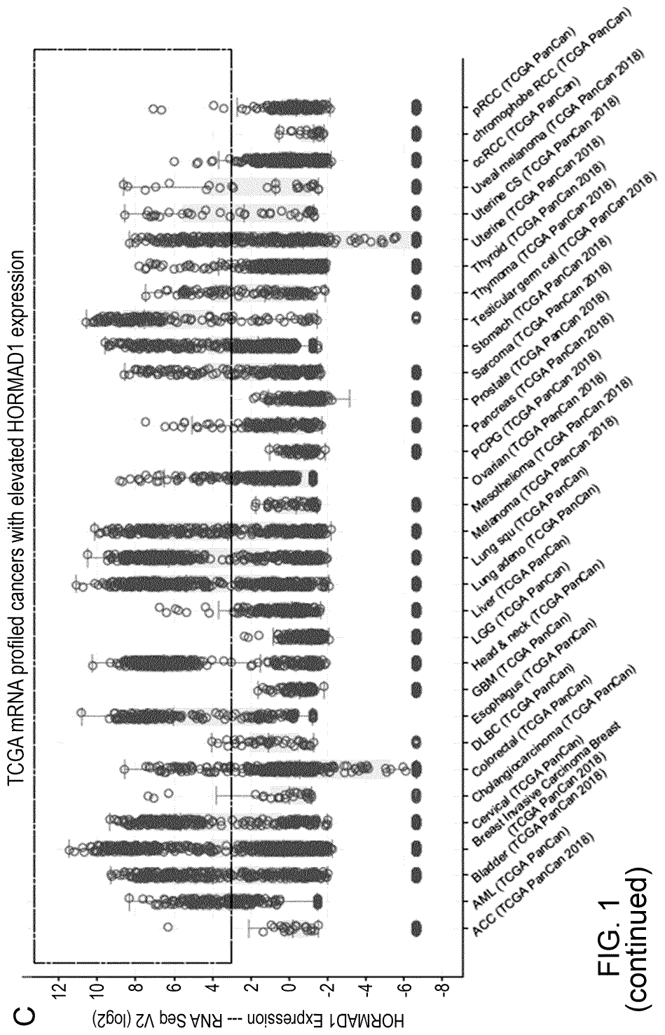

HORMAD1's normal physiological role and normal gene expression appears restricted to germ-line cells and HORMAD1 is not normally expressed in non-transformed somatic tissues. FIG. 1, A shows a consensus data set from the human protein atlas (www.proteinatlas.org) for HORMAD1. Consensus normalized expression (NX) levels is shown for 55 tissue types and 6 blood cell types, created by combining the data from the three transcriptomics datasets (HPA, GTEx and FANTOM5). It can be seen that the testes represents the major expression tissue with minor expression in skin, granulocytes, monocytes and dendritic cells. HORMAD1 is therefore expressed strongly in testis, weakly in placenta (not shown in A), with expression in other tissues less than 1% of that seen in the testis.

Using tumour mRNA expression data from the same source, HORMAD1 expression in human cancers was assessed. The results are shown in FIG. 1, B where it can be seen that HORMAD1 is expressed in a number of cancer histotypes. An analysis of tumour mRNA expression from large-scale tumour resequencing studies included in cbioportal (https://www.cbioportal.org) replicated this analysis and the results are shown in FIG. 1, C. These results are also discussed in Uhlen et al., 2015 and Uhlen et al., 2017.

Thus, HORMAD1 is expressed in certain cancer sub-types, including but not limited to breast cancers, such as triple negative (ER, PgR HER2 negative) and/or basal like breast cancers (60%), leukaemia, sarcomas, uveal melanomas, cholangiocarcinoma, melanomas, colorectal cancers, germ cell tumours of the testis and cancers of the bladder, cervix, oesophagus, head & neck, lung (including small cell or non-small cell lung cancer), ovary, pancreas, stomach, thyroid and uterus. In a preferred embodiment, the cancer is breast cancer, such as triple negative (ER, PgR HER2 negative) and/or basal like breast cancer.

It can therefore be seen that HORMAD1 is typically not expressed in normal tissue so an absence of HORMAD1 expression would be expected in normal, non-tumour, tissue. By contrast, the presence of HORMAD1 biomarkers—e.g. the expression of HORMAD1 in a sample is an effective diagnostic marker for certain cancers herein disclosed.

Accordingly, in one embodiment of the aspects described herein, the HORMAD1 positive cancer may be selected from breast cancer, preferably triple negative (ER, PgR HER2 negative) and/or basal like breast cancers, leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus. By expression of HORMAD1 is meant nucleic acid (e.g. RNA) or protein expression. Suitable biomarkers include: (i) HORMAD1 gene amplification (i.e. an increase in the number of copies of the HORMAD1 gene, preferably to two or above); (ii) presence of or elevated levels of HORMAD1 mRNA transcript; (iii) and/or presence of or elevated levels of HORMAD1 protein. Any one or more, or all of these biomarkers can be used to detect HORMAD1-positive cancers. Examples of HORMAD1-positive cancers are shown in FIG. 1 (panels B and C). In one example, HORMAD1 expression is determined by detecting the presence or absence of HORMAD1 protein or RNA in extra-cellular vesicles, for example in whole blood, serum or plasma. The expression of HORMAD1 may be detected by any suitable method.

We have previously reported that the aberrant expression of HORMAD1 in tumour cells perturbs the ability of cells to delay mitosis in response to improperly attached kinetochores through a weakening of the SAC and/or error correction by a mechanism that is independent of MAD2L1. Cells with a defective SAC are unable to maintain mitotic arrest for prolonged periods of time and undergo mitotic slippage sooner than cells with a functional SAC.

HORMAD1 exerts these effects by binding to Aurora B, disrupting the association with its co-factor, INCENP, and impairing the phosphorylation of Aurora B substrates. Consistent with this mechanism, aberrant expression of HORMAD1 drives cell sensitivity to either clinical MPS1 or Aurora kinase inhibitors and is synthetic lethal with depletion or small molecule inhibition of BUB1. BUB1 inhibition also sensitises patient-derived tumour organoids that over-express HORMAD1. In other words, we have shown that somatic cells that express HORMAD1 are hyper-dependent on the agents involved in mitosis, such as the agents mediating the SAC activation signal, including MPS1 and any residual Aurora B kinase, for cell fitness, proliferation and clonal survival.

As such, taken together, these data suggest that the out-of-context expression of HORMAD1 in tumour cells drives a weakening of the processes that control mitotic fidelity that contributes to chromosomal instability and induces dependency on a number of clinically relevant therapeutic targets. As such, tumoral HORMAD1 expression can act as a patient selection biomarker for synthetic lethal sensitivity to BUB1, Aurora B or MPS1 inhibitors.

The present invention has identified a further unexpected link between HORMAD1 expression and tumour cell-specific sensitivity/synthetic lethality to agents that generate DNA replication stress and/or inhibit pathways involved in DNA replication stress tolerance. These include agents involved in the inhibition of cell cycle checkpoint kinases, DNA translesion polymerases, and DNA polymerase theta (which has functions in DNA repair by micro-homology mediated end joining, also known as theta-mediated end joining (TMEJ)).

### Translesion Synthesis (TLS) Polymerases

Translesion synthesis (TLS) is a DNA damage tolerance pathway that allows cells to replicate DNA across DNA lesions, but has the potentially mutagenic effect of utilising low-fidelity DNA polymerases (Sale et al., 2012). Mammalian cells possess at least five TLS polymerases (Pol ζ [REV3L/REV7], REV1, POLH, POLK and POLI), each of which have different, but overlapping, substrate specificities (reviewed in Yang et al., 2018). In addition to their role in translesion bypass, TLS polymerases mediate replication fork restart in response to hydroxyurea-induced replication fork arrest (Tonzi et al., 2018). Importantly, TLS inhibition has been shown to modulate the therapeutic response to chemotherapy (Yamanaka et al., 2017; Wojtaszek et al., 2019; Zafar et al., 2018) and to the BRAF inhibitor Vemurafenib, in cells experiencing BRAFV600E oncogene-depletion induced stress (Temprine et al., 2020).

We presently demonstrate that expression of HORMAD1 increases the sensitivity of human cells, including cancer cells, to the knockdown or knockout of TLS polymerases. Therefore, in an example, the agent for use in the methods and treatments described herein modulates the expression and/or activity of one or more TLS polymerases, or one or more polymerases with TLS activity. Suitable small-molecule inhibitors targeting TLS polymerase inhibitors are described in Wojtaszek et al. (2019), Zafar et al. (2018) and Ketkar et al. (2019).

The Ensembl version number for REV3L may be ENSG00000009413. In one embodiment, REV3L may comprise or consist of SEQ ID NO: 2. REV3L may refer to a nucleic acid sequence comprising a sequence defined in NCBI Reference Sequence: NM_001372078.1.

The Ensembl version number for REV7 may be ENSG00000116670. In one embodiment, REV7 may comprise or consist of SEQ ID NO: 3. REV7 may refer to a nucleic acid sequence comprising a sequence defined in NCBI Reference Sequence: NM_006341.4.

The Ensembl version number for POLH may be ENSG00000170734. In one embodiment, POLH may comprise or consist of SEQ ID NO: 4. POLH may refer to a nucleic acid sequence comprising a sequence defined in NCBI Reference Sequence: NM_006502.3. POLH may refer to Gene ID: 5429 in the NCBI “Gene” resource.

The Ensembl version number for POLK may be ENSG00000122008. In one embodiment, POLK may comprise or consist of SEQ ID NO: 5. POLK may refer to Gene ID: 51426 in the NCBI “Gene” resource.

The Ensembl version number for POLI may be ENSG00000101751. In one embodiment, POLI may comprise or consist of SEQ ID NO: 6. POLI may refer to a nucleic acid sequence comprising a sequence defined in NCBI Reference Sequence: NM_007195.3.

The Ensembl version number for REV1 may be ENSG00000135945. In one embodiment, REV1 may comprise or consist of SEQ ID NO: 7. REV1 may refer to a nucleic acid sequence comprising a sequence defined in NCBI Reference Sequence: NM_016316.4.

### Polymerase Theta

TMEJ serves as an essential backup pathway to repair resected DSBs https://www.ncbi.nlm.nih.gov/pmc/articles/PMC8211653/-CR7 (Higgins GS, Boulton SJ. Beyond PARP-POLtheta as an anticancer target. Science. 2018;359:1217-1218). TMEJ is initiated by 5′ to 3′ resection factors, involves the poly-(ADP-ribose) polymerase PARP1, DNA ligase III and the eponymous 290 kDa Polymerase A family enzyme, DNA polymerase theta (Pole, encoded by POLQ) (Seki M, Marini F, Wood RD. POLQ (Pol theta), a DNA polymerase and DNA-dependent ATPase in human cells. Nucleic Acids Res. 2003;31:6117-6126). Pole possesses a N-terminal helicase-like domain and a C-terminal DNA polymerase domain separated by a non-structured central amino acid sequence (Newman JA, Cooper CDO, Aitkenhead H, Gileadi O. Structure of the helicase domain of DNA polymerase theta reveals a possible role in the microhomology-mediated end-joining pathway. Structure. 2015;23:2319-2330 and Malaby AW, Martin SK, Wood RD, Doublie S. Expression and structural analyses of human DNA polymerase theta (POLQ) Methods Enzymol. 2017;592:103-121) and is only found in multicellular organisms, where it is relatively well-conserved (Yousefzadeh MJ, Wood RD. DNA polymerase POLQ and cellular defense against DNA damage. DNA Repair (Amst.) 2013;12:1-9). The polymerase domain of Polθ includes three insertion amino acid loops, not conserved among other A-family DNA polymerases. It is this distinct structure that allows for the interaction, annealing, and extension of short single-stranded (ss)DNA primers (Kent T, Chandramouly G, McDevitt SM, Ozdemir AY, Pomerantz RT. Mechanism of microhomology-mediated end-joining promoted by human DNA polymerase theta. Nat. Struct. Mol. Biol. 2015;22:230-237 and Wyatt DW, et al. Essential roles for polymerase theta-mediated end joining in the repair of chromosome breaks. Mol. Cell. 2016;63:662-673). Biochemical studies have shown that the helicase domain of Polθ acts to displace RPA bound to the single-strand DNA overhang and facilitate annealing of short tracts of microhomology (>1-2 bp) that flank a DSB, potentially using distant DNA sites as templates (Seol JH, Shim EY, Lee SE. Microhomology-mediated end joining: Good, bad and ugly. Mutat. Res. 2018;809:81-87, Kent T, Chandramouly G, McDevitt SM, Ozdemir AY, Pomerantz RT. Mechanism of microhomology-mediated end-joining promoted by human DNA polymerase theta. Nat. Struct. Mol. Biol. 2015;22:230-237, van Schendel R, van Heteren J, Welten R, Tijsterman M. Genomic scars generated by polymerase theta reveal the versatile mechanism of alternative end-joining. PLoS Genet. 2016; 12: e1006368, Koole W, et al. A Polymerase Theta-dependent repair pathway suppresses extensive genomic instability at endogenous G4 DNA sites. Nat. Commun. 2014;5:3216). Polθ then employs its polymerase domain to initiate DNA synthesis to fill in the gaps, prior to ligation of the annealed DSB ends.

We presently demonstrate that expression of HORMAD1 increases the sensitivity of human cells, including cancer cells, to POLQ knockdown or POLQ inhibition using previously described small molecule inhibitors (Zhou J, Gelot C, Pantelidou C, Li A, Yücel H, Davis RE, Farkkila A, Kochupurakkal B, Syed A, Shapiro GI, Tainer JA, Blagg BSJ, Ceccaldi R, D'Andrea AD. Nat Cancer. 2021 Jun;2 (6):598-610). Therefore, in an example, the agent for use in the methods and treatments described herein modulates the expression and/or activity of POLQ.

It has been reported that HORMAD1 expression causes genomic instability in breast cancer and mouse embryonic stem (ES) cell lines, at least in part, through modest effects on Rad51-mediated homologous recombination (HR) and non-homologous end-joining (NHEJ) (Watkins et al., 2015). The effects of HORMAD1 on HR appear to be context dependent, however, and others have found evidence that HORMAD1 can in fact enhance HR (Gao et al., 2018; Liu et al., 2020; Nichols et al., 2018; Wang et al., 2018). Therefore, and without wishing to be bound by theory, it is hypothesised that aberrant HORMAD1 expression induces hyper-dependency upon POLQ activity by causing a dependency on TMEJ and/or translesion synthesis.

In an example, the agent that modulates the activity of DNA polymerase theta of the present invention include DNA polymerase theta inhibitors.

Suitable DNA polymerase theta inhibitors include aminocoumarins or pharmaceutically acceptable salts thereof. Aminocoumarins include Novobiocin, Coumermycin, and Clorobiocin. In an example, the DNA polymerase theta inhibitor is Novobiocin or a pharmaceutically acceptable salt thereof (Zhou J, Gelot C, Pantelidou C, Li A, Yücel H, Davis RE, Farkkila A, Kochupurakkal B, Syed A, Shapiro GI, Tainer JA, Blagg BSJ, Ceccaldi R, D'Andrea AD. Nat Cancer. 2021 Jun;2(6):598-610). Suitable DNA polymerase theta inhibitors also include ART558 (CAS Number: 2603528-97-6) and ART812 (available from Artios Pharma Limited, Cambridge, UK and MedChem Express and described in Zatreanu et al Nat Commun. 2021 Jun. 17;12(1): 3636), or pharmaceutically acceptable salts thereof.

In one embodiment, the structure of ART558 may be as follows:

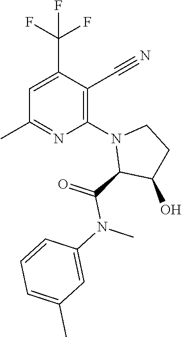 [SMILES: Cc1cccc(N(C)C(=O)[C@@H]2[C@H](O)CCN2c2nc(C)cc(C(F)(F)F)c2C#N)c1]

The Ensembl version number for POLQ may be ENSG00000051341. In one embodiment, POLQ may comprise or consist of SEQ ID NO: 8. POLQ may refer to a nucleic acid sequence comprising a sequence defined in NCBI Reference Sequence: NM_199420.4. POLQ may refer to Gene ID: 10721 in the NCBI “Gene” resource.

### ATR/CHK1

ATR (ataxia telangiectasia and Rad3-related) is one of the apical kinases of the DNA damage response. The ATR kinase complex is critical for recognising and triggering a response to replication stress (RS), a collection of phenotypes that describe abnormal replication fork function e.g. fork slowing, stalling, collapse or an increase in replication fork speed (Zekman et al., 2014). RS is common in cancer; for example, the increase in replication that results from oncogene activation (e.g. via Myc or Cyclin E upregulation) is a well-established cause of RS (Halazonetis et al., 2008; Luo et al., 2009). In response to abnormal fork progression, ATR, along with its binding partner ATRIP, is recruited to the extended tracts of RPA-coated single-strand DNA (ssDNA) that often form at dysregulated forks (Zou et al., 2003; MacDougall et al., 2007). RPA-bound ATR is then trans-activated by TOPBP1 (Kumagai et al., 2006) or ETAA1 (Bass et al., 2016; Haahr et al., 2016), which leads ATR to phosphorylate and activate downstream effectors including the kinase CHK1. These effectors stall the cell cycle, mediate DNA repair, prevent apoptosis and limit the firing of latent replication origins, which could otherwise exacerbate RS (Berti et al., 2016). In totality, this ATR-mediated RS response allows cells to repair and restart replication forks so that replication can be completed before DNA is divided between daughter cells. When ATR is partially inhibited, which can be achieved via drug-like small molecule kinase inhibitors (ATRi), the normal response to RS is impaired (Wagner et al., 2016). ATRi elicit anti-tumour effects in both pre-clinical cancer model systems (Karnitz et al., 2015) and in early phase clinical trials (Yap et al., 2020), without eliciting severe, non-tumour toxicity; this is presumably because ATR inhibition exacerbates pre-existing tumour cell-specific RS to the point where tumour cells are not viable.

We presently demonstrate that expression of HORMAD1 increases the sensitivity of human cells, including cancer cells, to ATR knockdown and CHK1 inhibition using previously described small molecule inhibitors. Therefore, in an example, the agent for use in the methods and treatments described herein inhibits the ATR/CHK1 pathway. In particular, the agent may modulate the expression and/or activity of enzymes involved in the ATR/CHK1 pathway, in particular ATR and/or CHK1. Examples of suitable agents may include PF47736 (CAS Number: 952021-60-2) available from Sigma Aldrich, SAR020106 (CAS Number: 1184843-57-9) available from Selleckchem.com, and LY2606368 (CAS Number: 1234015-52-1) available from Selleckchem.com.

The Ensembl version number for ATR may be ENSG00000175054. In one embodiment, ATR may comprise or consist of SEQ ID NO: 9. ATR may refer to a nucleic acid sequence comprising a sequence defined in NCBI Reference Sequence: NM_001184.4.

The Ensembl version number for CHK1 may be ENSG00000149554. In one embodiment, CHK1 may comprise or consist of SEQ ID NO: 10. CHK1 may refer to a nucleic acid sequence comprising a sequence defined in NCBI Reference Sequence: NM_001114122.3.

In an example, the agent for use in the methods and treatments described herein may, alternatively or additionally, inhibit other cell-cycle (e.g. S-phase) checkpoint kinases.

### TDP1

Tyrosyl DNA phosphodiesterase 1 (TDP1) has roles in DNA replication stress tolerance and repair.

We presently demonstrate that expression of HORMAD1 increases the sensitivity of human cells, including cancer cells, to TDP1 knockdown. Therefore, in an example, the agent for use in the methods and treatments described herein modulates the expression and/or activity of TDP1. Suitable TDP1 inhibitors include: sodium orthovanadate; tungstate; furamidine; neomycin B; and NSC88915 (4-Pregnen-21-ol-3,20-dione-21-(4-bromobenzenesulfonate)). Suitable TDP1 inhibitors are also described in Il'ina et al (2020) and Pommier et al. (2014).

The Ensembl version number for TDP1 may be ENSG00000042088. In one embodiment, TDP1 may comprise or consist of SEQ ID NO: 11. TDP1 may refer to a nucleic acid sequence comprising a sequence defined in NCBI Reference Sequence: NM_018319.4.

### BRIP1

BRCA1-interacting helicase 1 (BRIP1, also known as BACH1), interacts with BRCA1 and is involved in double-strand DNA-break repair.

We presently demonstrate that expression of HORMAD1 increases the sensitivity of human cells, including cancer cells, to BRIP1 knockdown. Therefore, in an example, the agent for use in the methods and treatments described herein modulates the expression and/or activity of BRIP1.

The Ensembl version number for BRIP1 may be ENSG00000136492. In one embodiment, BRIP1 may comprise or consist of SEQ ID NO: 12. BRIP1 may refer to a nucleic acid sequence comprising a sequence defined in NCBI Reference Sequence: NM_032043.3.

### XRCC1

DNA repair protein XRCC1, or X-ray repair cross-complementing protein 1, (encoded by the XRCC1 gene), is involved in the DNA single-strand break repair.

We presently demonstrate that expression of HORMAD1 increases the sensitivity of human cells, including cancer cells, to XRCC1 knockdown. Therefore, in an example, the agent for use in the methods and treatments described herein modulates the expression and/or activity of XRCC1.

The Ensembl version number for XRCC1 may be ENSG00000073050. In one embodiment, XRCC1 may comprise or consist of SEQ ID NO: 13. XRCC1 may refer to a nucleic acid sequence comprising a sequence defined in NCBI Reference Sequence: NM_006297.3.

### Compounds & Compositions

Compounds of the present invention or medicaments comprising the same can be prepared for administration using methodology well known in the pharmaceutical art. Examples of suitable pharmaceutical formulations and carriers are described in “Remington's Pharmaceutical Sciences” by E. W. Martin.

Suitable examples of the administration form of compounds of the present invention or a pharmaceutically acceptable salt thereof include without limitation oral, topical, parenteral, sublingual, rectal, vaginal, ocular, and intranasal. Parenteral administration includes subcutaneous injections, intravenous, intramuscular, intrasternal injection or infusion techniques.

Pharmaceutical compositions of the invention can be formulated so as to allow a compound according to the present invention to be bioavailable upon administration of the composition to an animal, preferably human. Compositions can take the form of one or more dosage units, where for example, a tablet can be a single dosage unit, and a container of a compound according to the present invention may contain the compound in liquid or in aerosol form and may hold a single or a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example inhalatory administration. Powders may also be used for inhalation dosage forms. The term “carrier” refers to a diluent, adjuvant or excipient, with which the compound according to the present invention is administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In one embodiment, when administered to an animal, the compounds and compositions according to the present invention, and pharmaceutically acceptable carriers are sterile. Water is a preferred carrier when the compounds according to the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more for the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agent such as sucrose or saccharin; a flavoring agent such as peppermint, methyl salicylate or orange flavoring; and a coloring agent.

When the composition is in the form of a capsule (e.g. a gelatin capsule), it can contain, in addition to materials of the above type, a liquid carrier such as polyethylene glycol, cyclodextrins or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The following examples further illustrates the invention. They should not be interpreted as a limitation of the scope of the invention.

### EXAMPLES

### siRNA Screening Identifies Candidate HORMAD1-Induced Gene Dependencies

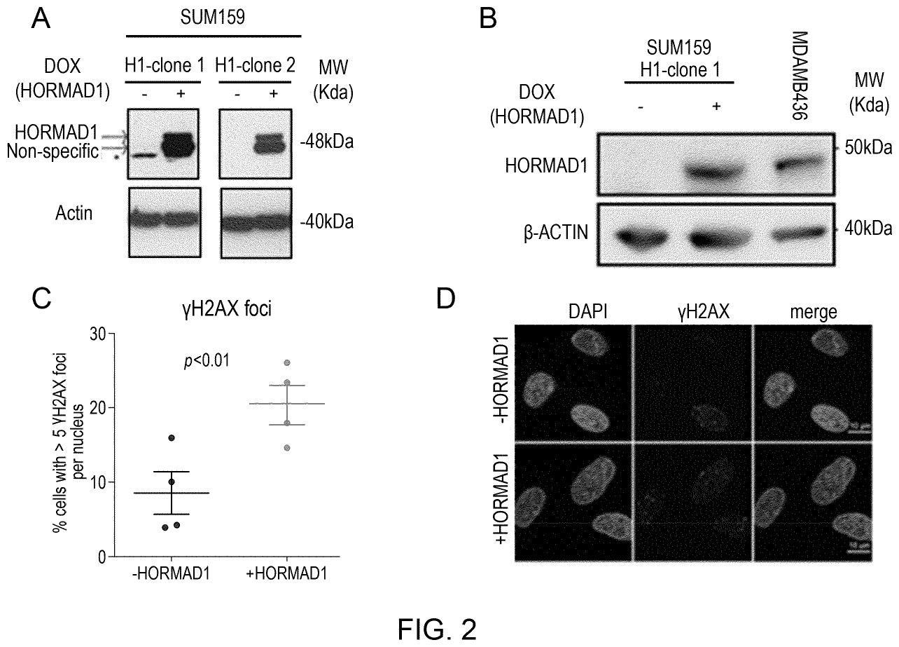

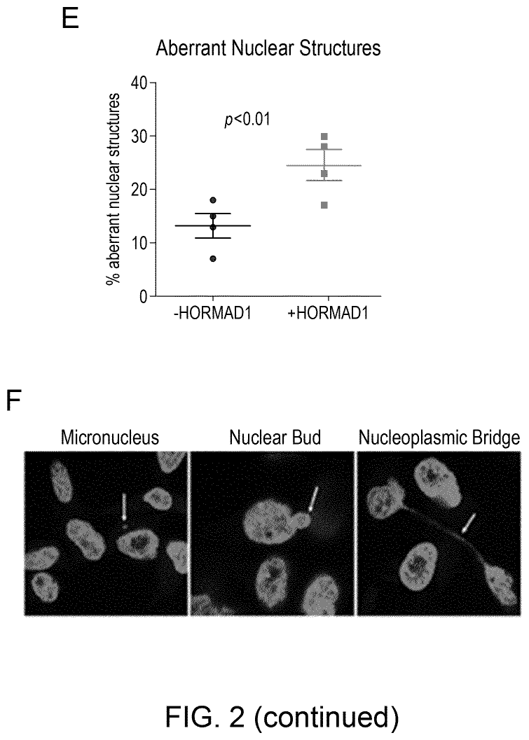

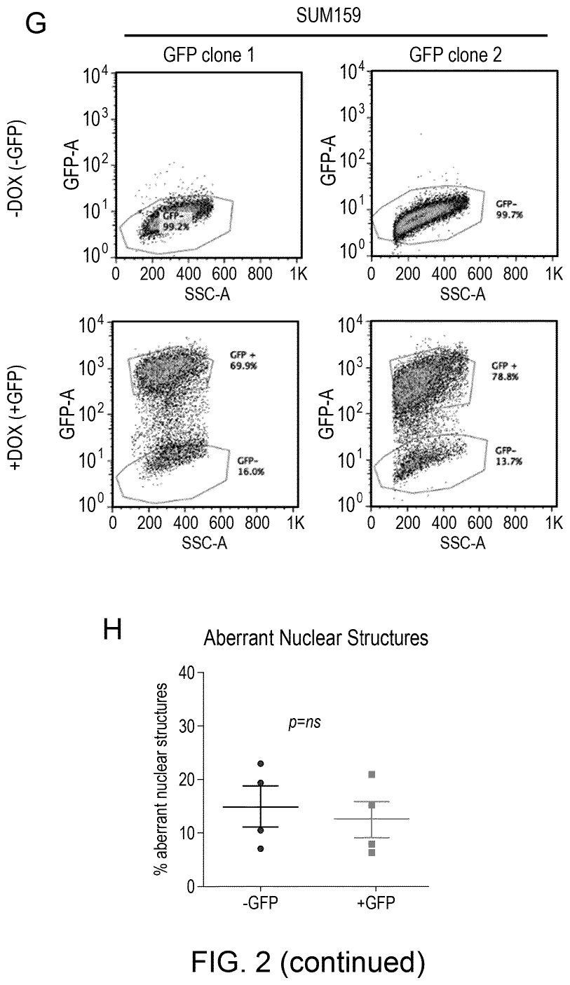

To identify genetic dependencies associated with illegitimate HORMAD1 expression we generated SUM159 cell lines that expressed inducible high levels of HORMAD1 when exposed to doxycycline. We selected SUM159 cells for this purpose as: (i) this cell line was derived from a TNBC and possesses a pathogenic p53 mutation, making this relevant to the TNBC context we wished to understand; (ii) SUM159 cells lack endogenous HORMAD1 expression (Watkins et al., 2015; Gao et al., 2018); and (iii) SUM159 cells were known to be amenable to siRNA screening (Brough et al., 2011). To generate a controlled experimental system, we performed single cell cloning of SUM159 cells prior to and post transduction of an inducible expression construct in a pINDUCER20-HORMAD1 lentivirus (Meerbrey et al., 2011), and selected two clones for further experiments. We confirmed doxycycline-induced expression of HORMAD1 in these clones and also showed that the HORMAD1 levels achieved in these models is comparable to that found in the endogenous HORMAD1 expressing breast cancer line mdamb436 (FIGS. 2, A & B). In our SUM159 clones, induction of HORMAD1 increased the proportion of nuclei with >5 γH2AX foci (FIGS. 2, C and D) and increased the number of aberrant nuclear structures, namely micronuclei, nuclear buds and nucleoplasmic bridges compared to control SUM159 engineered with a pINDUCER20-GFP, which allowed expression of GFP upon doxycycline induction (FIGS. 2, E-H).

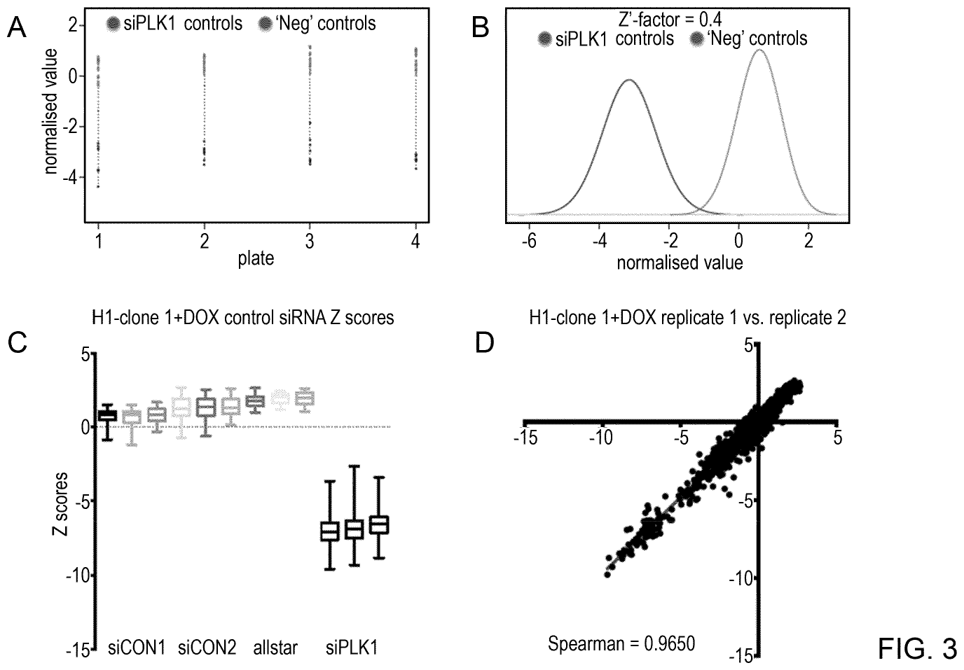

We then performed siRNA screening in one HORMAD1-expressing isogenic SUM159 clone (H1-clone 1), as well as in the corresponding parental SUM159 cell line. Our siRNA library targeted 1280 genes with pools of 4 siRNAs, which included 720 genes encoding the human kinome and kinase-related genes, 80 tumour suppressor genes, and 480 genes featuring in the Cancer Gene Census list (Futreal et al., 2004). Details related to the siRNA library were published elsewhere (Jones et al., 2017). For the screen, cells were reverse-transfected with the siRNA library in 384-well plates. Twenty-four hours after transfection replica plates were exposed either to doxycycline, to induce HORMAD1 expression, or to the doxycycline vehicle, DMSO. Cell viability was estimated five days post-transfection using CellTiter-Glo. In order to compare between different experimental arms, cell viability data were first converted into Z-scores and quality control assessments conducted as described previously (Campbell et al., 2016; Lord et al., 2008) (FIG. 3). To identify genetic dependencies induced by HORMAD1 expression, we used an analytical approach commonly used in siRNA screens to identify drug sensitisation effects (Lord et al., 2008), drug effect (DE) Z scores, which allowed the effect of each siRNA on cell viability to be compared in the presence and absence of doxycycline/HORMAD1 expression. DE-Z scores were calculated for each siRNA for both H1-clone 1 and parental SUM159 cells. In this case, negative DE Z-scores indicated that HORMAD1 expression caused sensitivity to the siRNA. As the Z-3 threshold is roughly equivalent to three standard deviations from the median effect, we considered siRNAs with a DE-Z score<−3 in H1-clone 1 and >−2 in parental SUM159 cells as candidate HORMAD1-related genetic dependencies. As an additional filter, we removed siRNAs which, in the absence of doxycycline caused profound cell growth inhibition (Z<−3), as this suggests they target a core essential gene and cause common artefacts in such screens.

### Validation of HORMAD1-Induced DNA Damage Response Genetic Dependencies

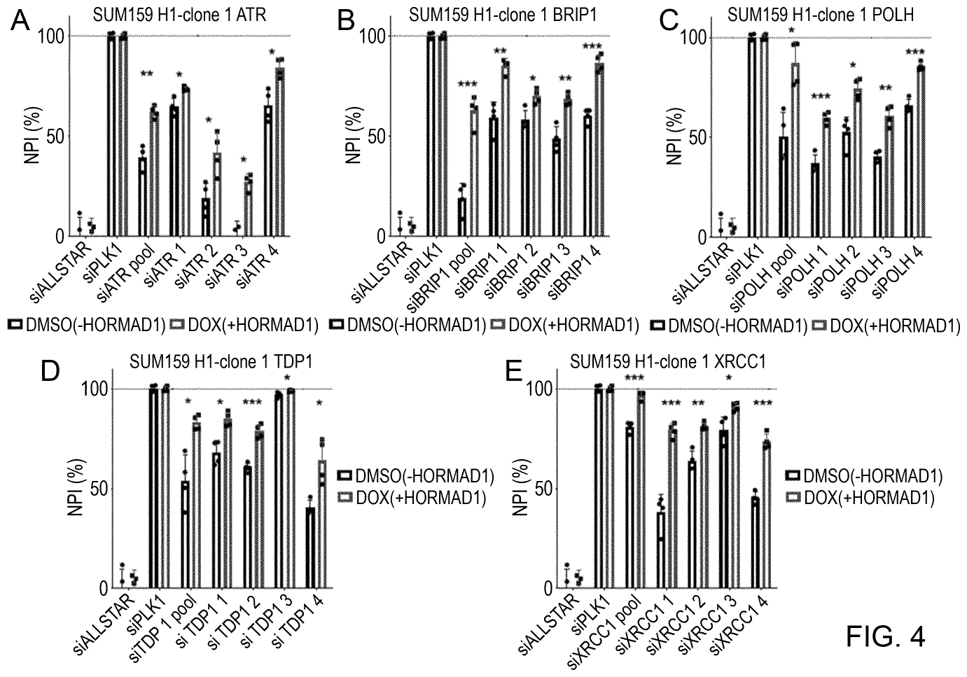

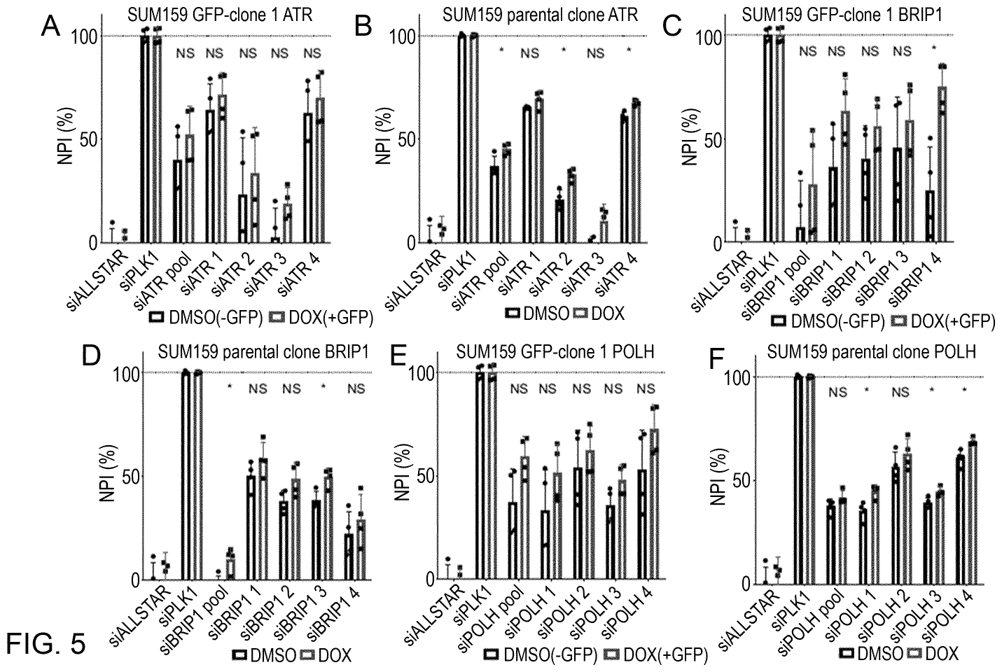

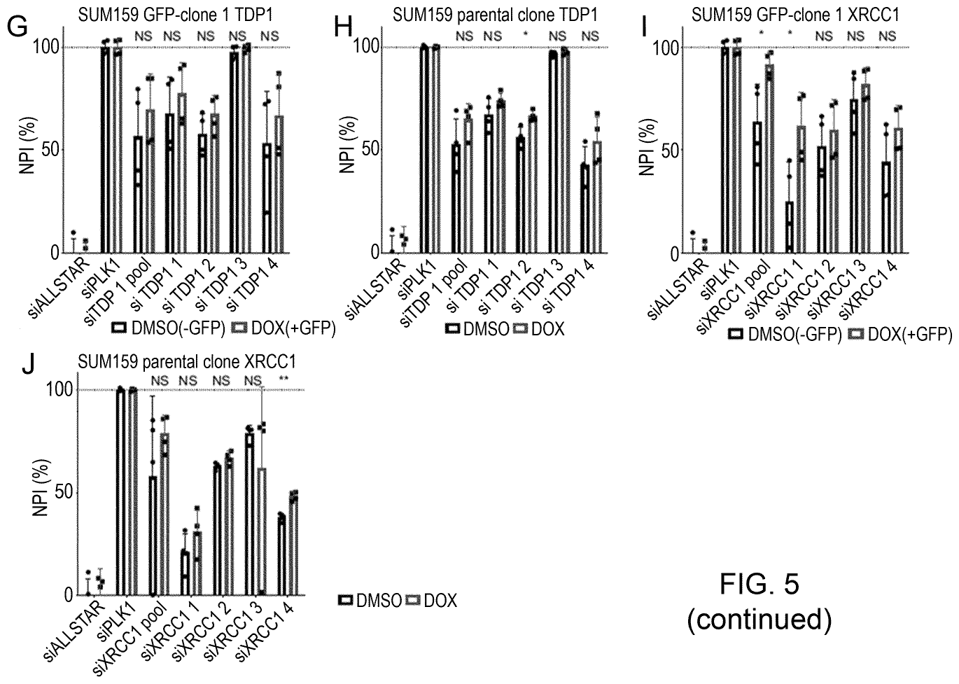

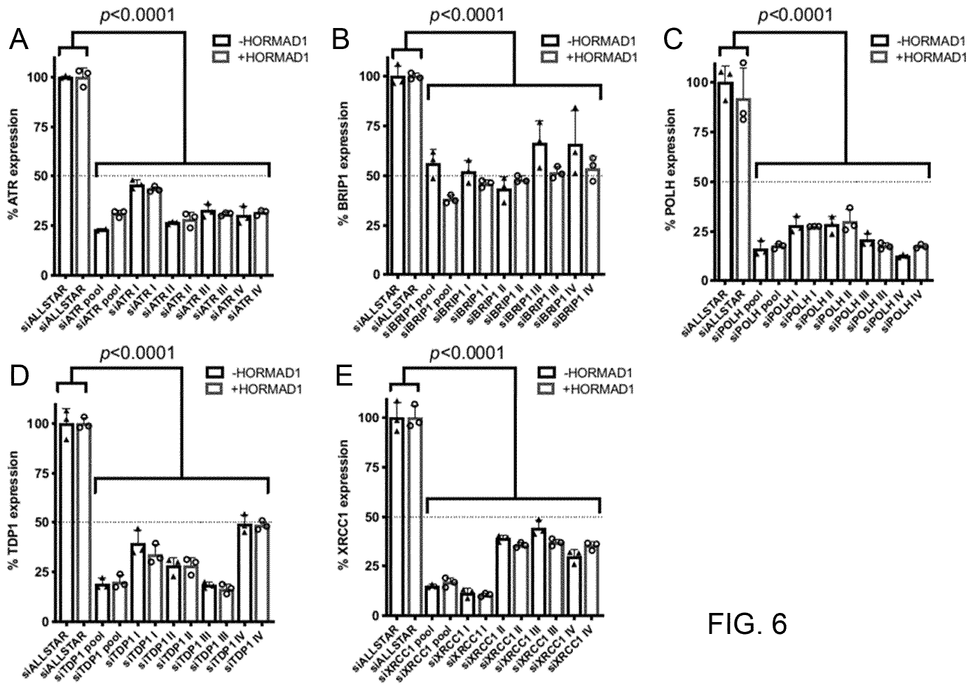

To exclude further analysis of “off-target” effects of RNAi, we performed a secondary validation screen using four individual siRNA oligonucleotides. The secondary validation screen was performed in three cell lines: the HORMAD1-inducible isogenic SUM159 clone, the parental SUM159 clonal cell line from the original screen and an additional SUM 159 isogenic clone with doxycycline-inducible expression of GFP, used as a means to assess the possibility that the plnducer vector expression system and/or doxycycline exposure alone caused genetic dependencies. GFP induction in this system had not led to an increase in the number of aberrant nuclear structures, suggesting it would be an appropriate negative-control model (FIGS. 2, G and H). Gene effects were considered ‘on-target’ if two or more of the individual siRNAs present in the original siRNA pool resulted in significant doxycycline-induced cell inhibitory effects in the HORMAD1-expressing line. In addition, we excluded genes for which the same siRNAs resulted in doxycycline-induced cell inhibitory effects in both GFP-expressing and parental doxycycline-treated cells, as these were likely to represent sensitising effects of doxycycline or associated effects of exogenous protein expression itself. Finally, we confirmed the efficacy of each siRNA oligonucleotide and siRNA pool using RT-qPCR analysis. According to these criteria, the following genes were validated as “on-target” HORMAD1-induced genetic dependencies: ATR, BRIP1, POLH, TDP1 and XRCC1 (FIG. 4; FIG. 5). For the validated genes, all siRNAs resulted in at least 30% gene knockdown (FIG. 6).

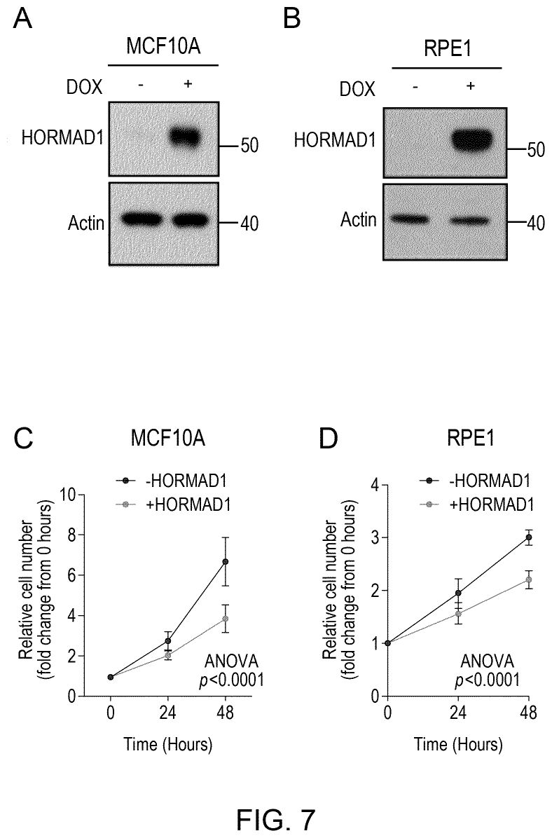

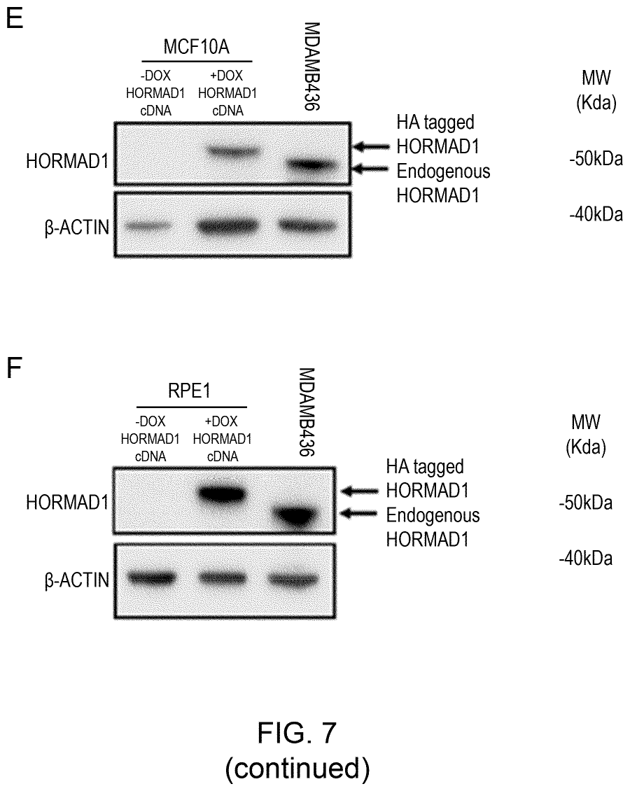

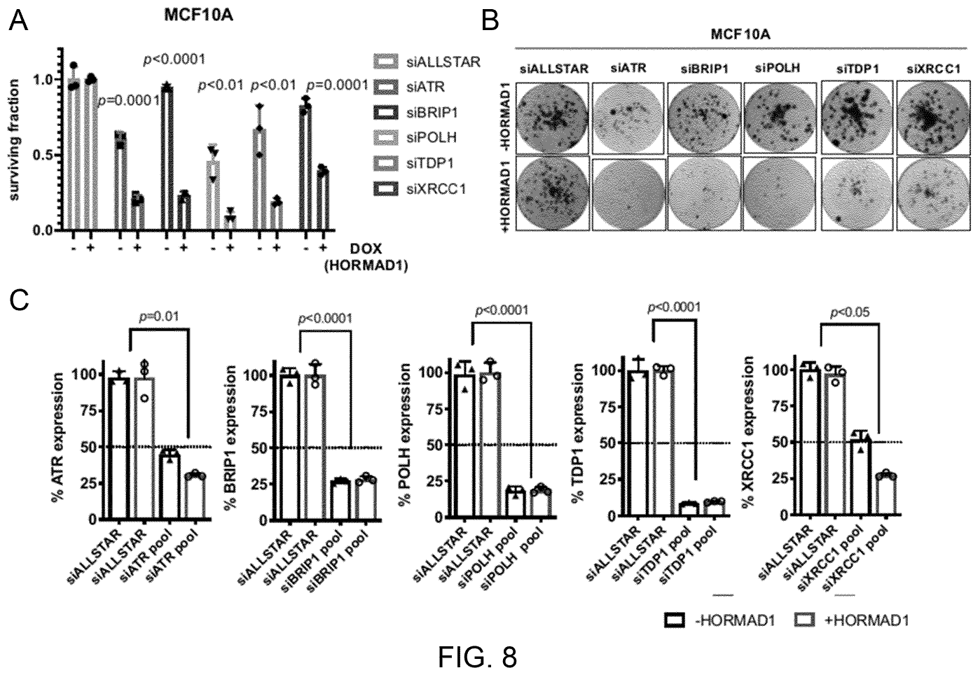

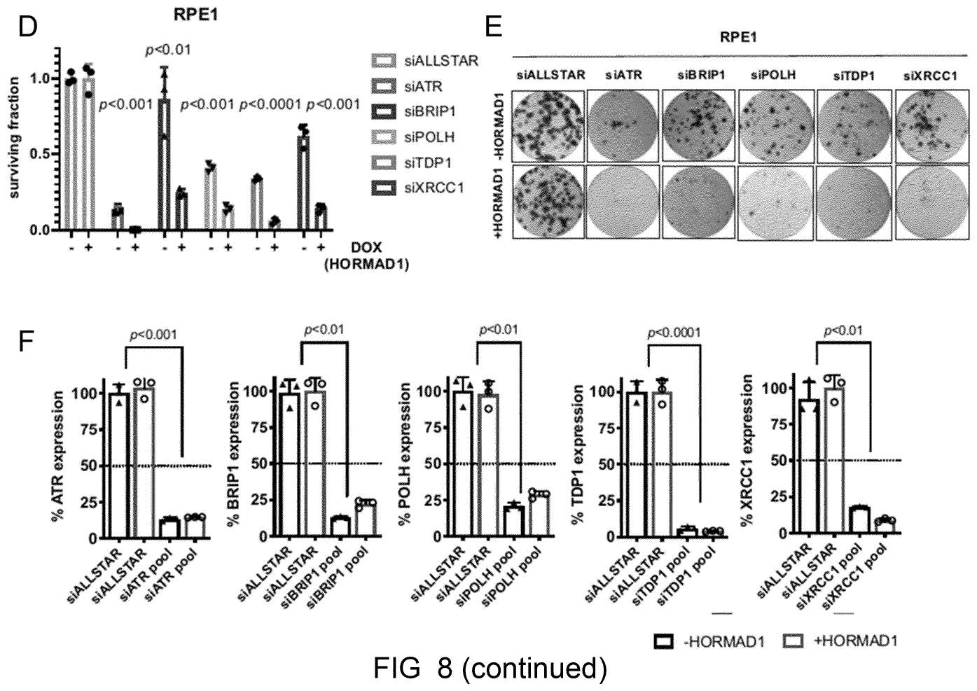

Next, we investigated whether these genetic dependencies were specific or “private” to the genetic background of SUM159 cells, or whether they represented more penetrant (Ryan et al., 2018) HORMAD1-driven dependencies using isogenic doxycycline-inducible HA tagged-HORMAD1 expressing models of the non-transformed cell lines MCF10A and RPE1 (FIGS. 7, A, B, E and F). In these lines expression levels of HA tagged HORMAD1 were comparable to those seen in the HORMAD1 positive breast cancer cell line MDAMB436. Interestingly, time-lapsed microscopy of these cells revealed that HORMAD1 impaired cellular growth (FIGS. 7, C and D), which is consistent with the observation that HORMAD1 expression in somatic cells drives induction of DNA damage with consequent genomic instability. Using clonogenic survival assays, we observed significant and HORMAD-1-specific reduction in single-cell colony-formation capacity, exacerbated by ATR, BRIP1, POLH, TDP1 and XRCC1 depletion in both systems (FIGS. 8, A-F). Our validation experiments suggested that ATR, BRIP1, POLH, TDP1 and XRCC1 genetic dependencies operated in multiple model systems.

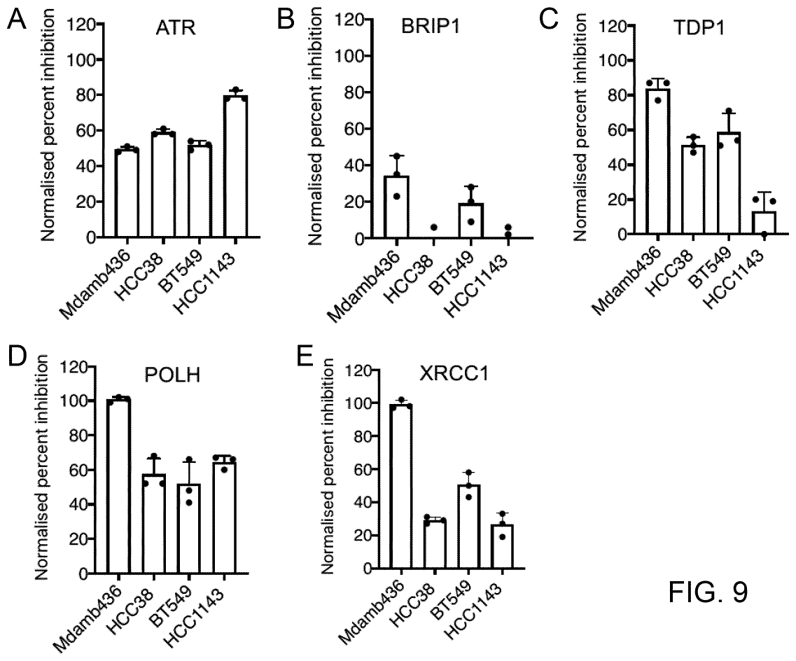

Having identified HORMAD1 induced dependencies in isogenic cell line models we next performed siRNA mediated knockdown experiments in the HORMAD1 positive cell lines HCC38, BT549 HCC1143 and MDAMB436 for ATR, BRIP, POLH, TDP1, XRCC1 (FIG. 9). POLH knockdown led to >50% cell inhibition in all four cells lines (FIG. 9, D), while both ATR and TDP1 knockdown led to >50% cell inhibition in three of the four cells lines tested (FIGS. 9, A and C). This data supports the idea that POLH, TDP1 and ATR represent penetrant sensitivities for HORMAD1 expressing cells.

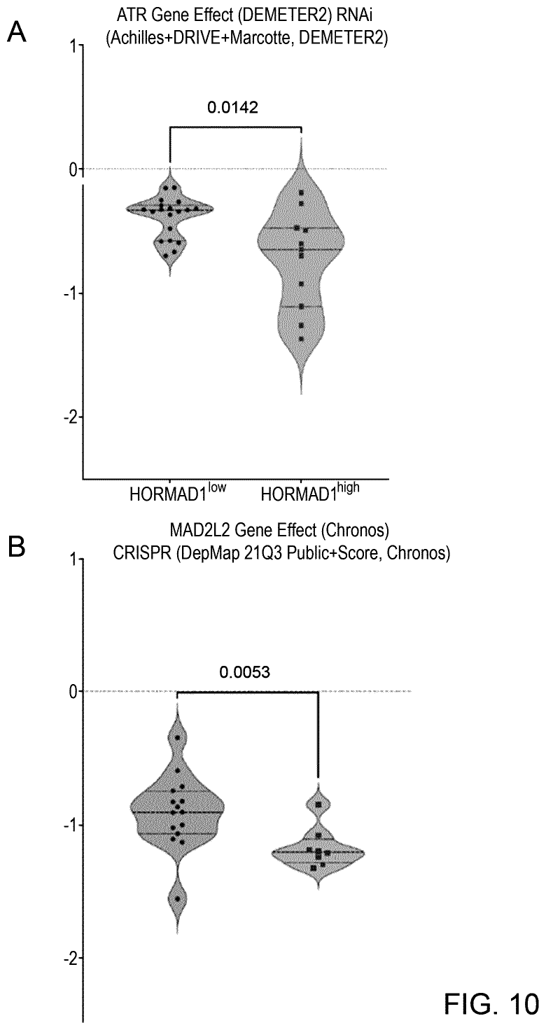

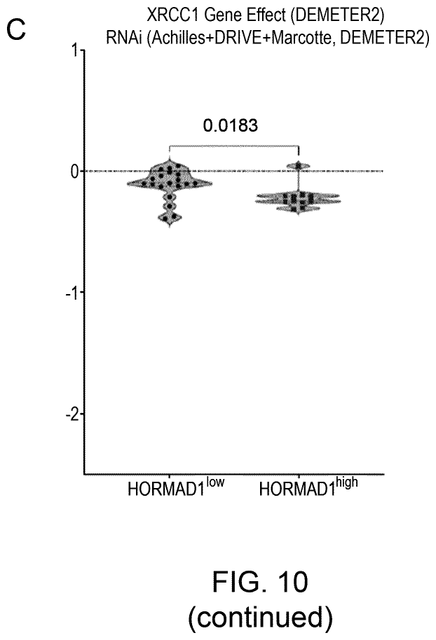

We also analysed RNAi and CRISPR data from Depmap (McDonald et al., 2017). This analysis allowed us to compare sensitivity between HORMAD1 positive and negative cell lines and showed that ATR (p=0.0142) and to a lesser extent XRCC1 (p=0.0183), showed greater dependency in HORMAD1 positive breast cancer cell lines as observed by siRNA mediated knockdown (FIG. 10). HORMAD1 positive cell lines also showed a greater dependency to REV7 (also known as MAD2L2) in the CRISPR dataset (FIG. 10, B). Given our observation that all the HORMAD1 expressing breast cancer cell lines showed sensitivity to POLH knockdown, we further investigated how the silencing of POLH and a wider group of TLS polymerases, affected the viability of HORMAD1-expressing cells.

### Orthogonal Validation of POLH as a HORMAD1-Induced Genetic Dependency

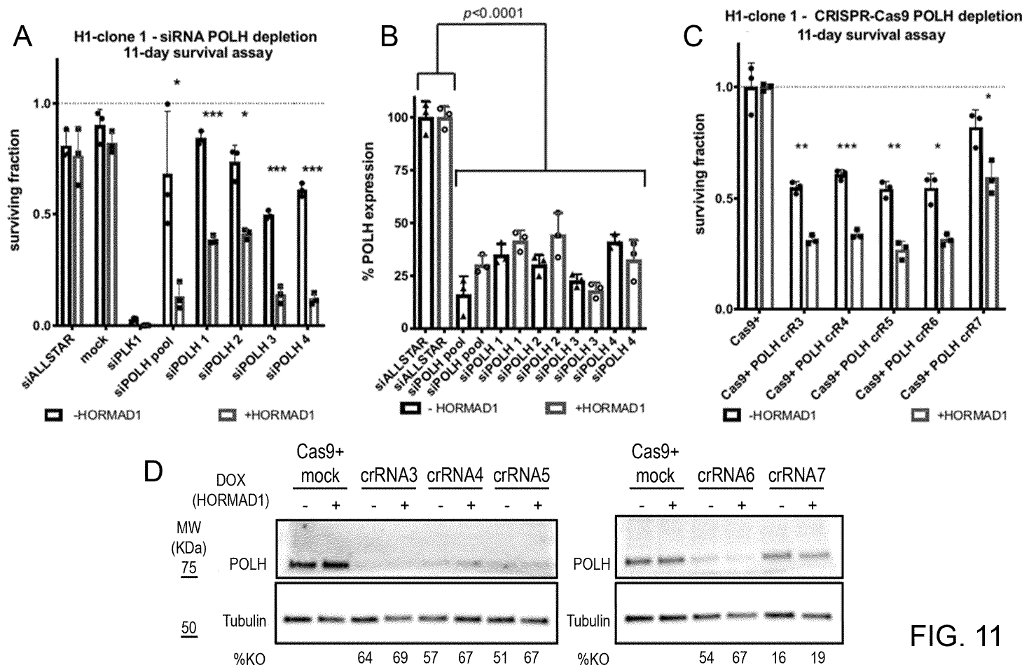

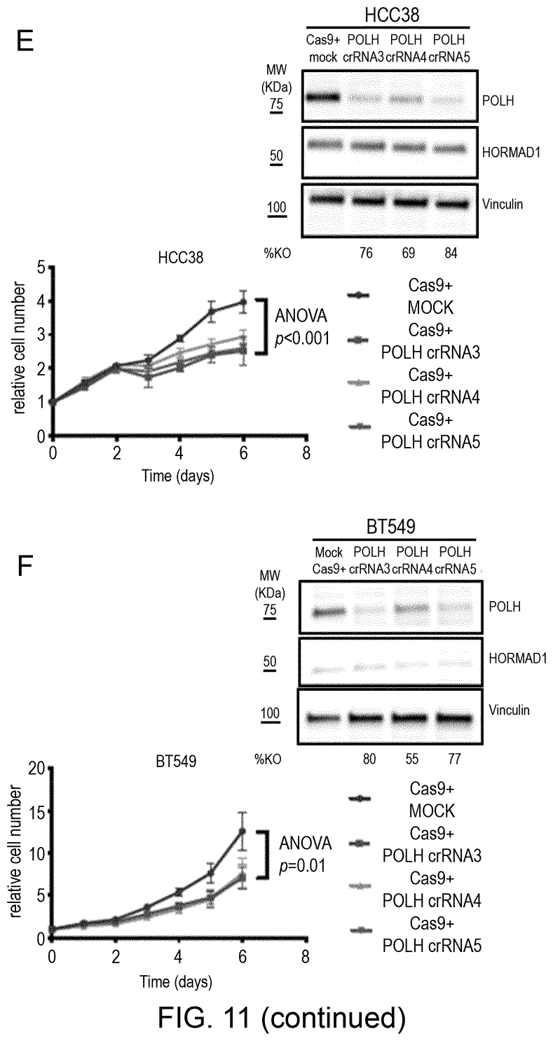

As our screen had been conducted in the context of an acute 5-day exposure to HORMAD1 we wished to assess whether dependency upon POLH occurred in SUM159cells adapted to expressing HORMAD1 over a longer time period. Both longer-term expression of HORMAD1 (14 days in total) and continuous HORMAD1 expression for 21.5 weeks resulted in a significant decrease in cellular viability following siRNA-mediated depletion of POLH, confirmed by RT-qPCR (FIGS. 11, A-B; FIGS. 12, A-B). Given the potential off-target effects of siRNA transfections, we sought to validate on-target POLH sensitivity using the orthogonal technique of Edit-R CRISPR-Cas9 mediated gene editing to deplete the wild-type POLH gene product. The effect of HORMAD1 on cellular sensitivity to POLH depletion was confirmed 11 days after guide transfection (FIGS. 11, C-D). Finally, we investigated whether POLH depletion would inhibit cellular growth in two TNBC cell lines expressing endogenous HORMAD1, namely HCC38 and BT549. By tracking cell population growth with Incucyte microscopy, we found that both models displayed reduced cellular growth following POLH editing (FIGS. 11, E, F), despite the limitations of variable Edit-R guide and CRISPR-Cas9 transfection efficiency and consequent incomplete gene editing within a bulk transfected population. Taken together, our data demonstrate that HORMAD1 expression leads to a dependency on the TLS polymerase POLH that is not private to the SUM159 model system in which it was first discovered.

### HORMAD1 Expression Leads to a Functional Dependency on Multiple Translesion Synthesis Proteins

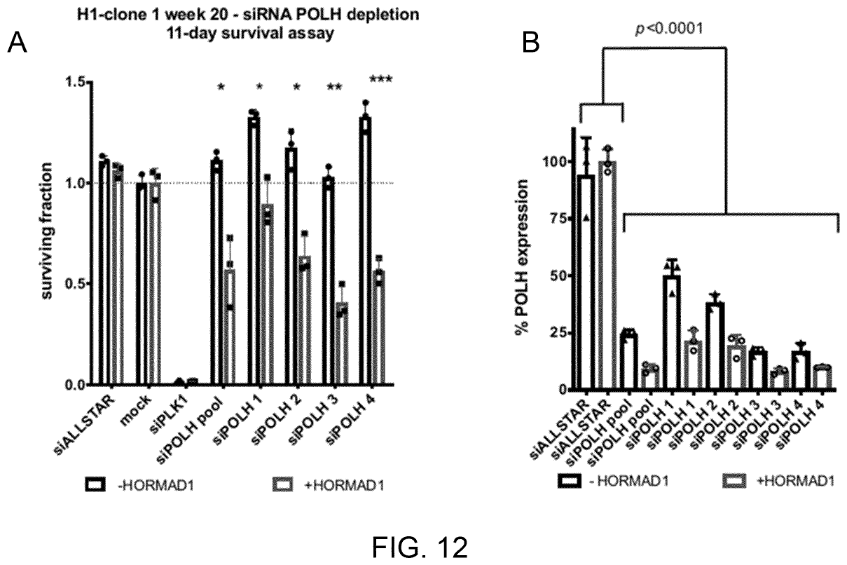

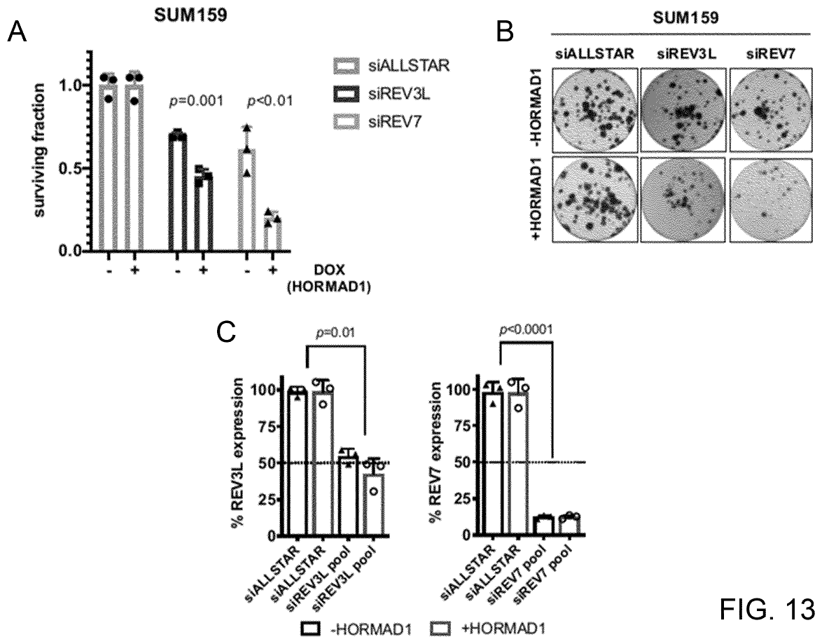

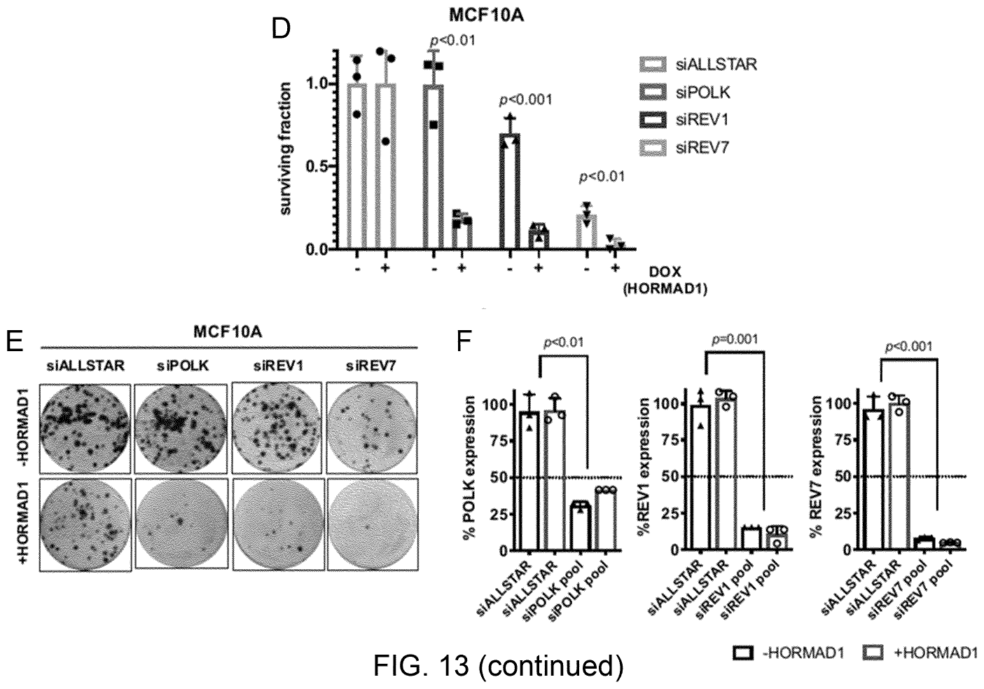

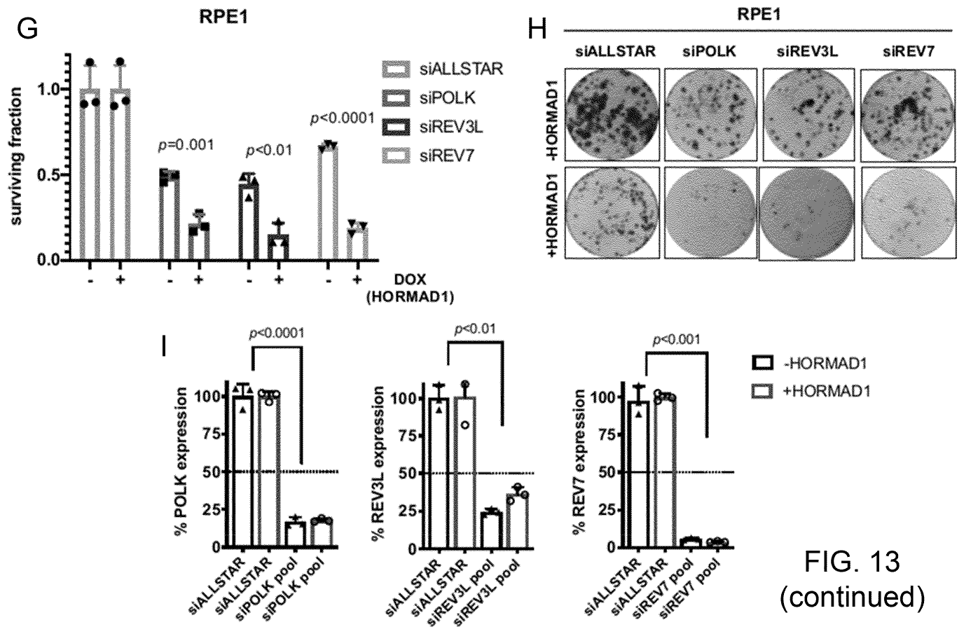

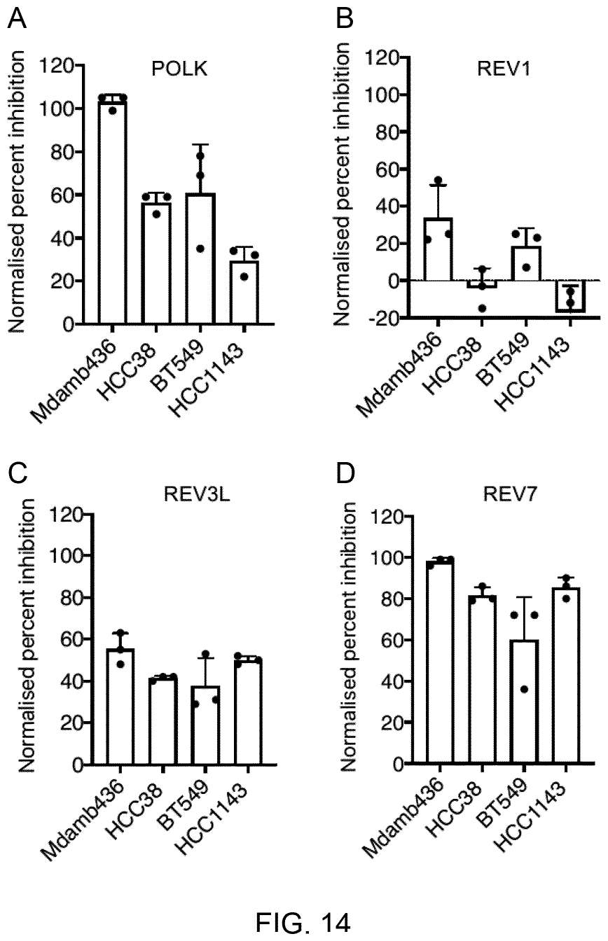

POLH is a TLS polymerase that facilitates replication across replication-blocking DNA lesions (Sale et al., 2012). As a wider group of TLS polymerases are involved in similar functions, we hypothesised that the observed HORMAD1-driven POLH dependency could extend to additional TLS polymerases. To test this, we depleted POLI, POLK, REV1, REV3L and REV7 using siRNA and used clonogenic survival assays to test effects on clonogenic capacity following inducible HORMAD1 expression in SUM159, MCF10A and RPE1. These experiments revealed that REV7 depletion impaired clonogenic survival to a greater extent in HORMAD1-expressing SUM159 (FIGS. 13, A-C), MCF10A (FIGS. 13, D-F) and RPE1 (FIGS. 13, G-I) cells. We also observed a HORMAD1-driven sensitivity to REV3L in SUM159 (FIGS. 13, A-C) and RPE1 (FIGS. 13, G-I), to POLK in MCF10A (FIGS. 13, D-F) and RPE1 (FIGS. 13, G-I), and to REV1 in MCF10A (FIGS. 13, D-F). We next performed siRNA mediated knockdown experiments in the HORMAD1 positive cell lines HCC38, BT549 HCC1143 and MDAMB436 for POLK, REV1, REV3L and REV7 (FIG. 14). We found that REV7 produced cell inhibition of >50% in all four lines tested (FIG. 14, D). POLK knockdown produced cell inhibition of >50% in three out of four lines tested (FIG. 14, A). HORMAD1 positive cell lines also showed a greater dependency to REV7 (also known as MAD2L2) in depmap CRISPR screens (FIG. 10, B). This data supports the idea that TLS polymerases represent penetrant sensitivities in HORMAD1 expressing cells.

Taken together, our results reveal a number of genes that are essential for cellular viability following out-of-context expression of HORMAD1 and suggest that TLS may enable replication stress tolerance in cells expressing HORMAD1.

### Dox-Induced Expression of HORMAD1 Increases Cellular Genetic Addiction to POLQ

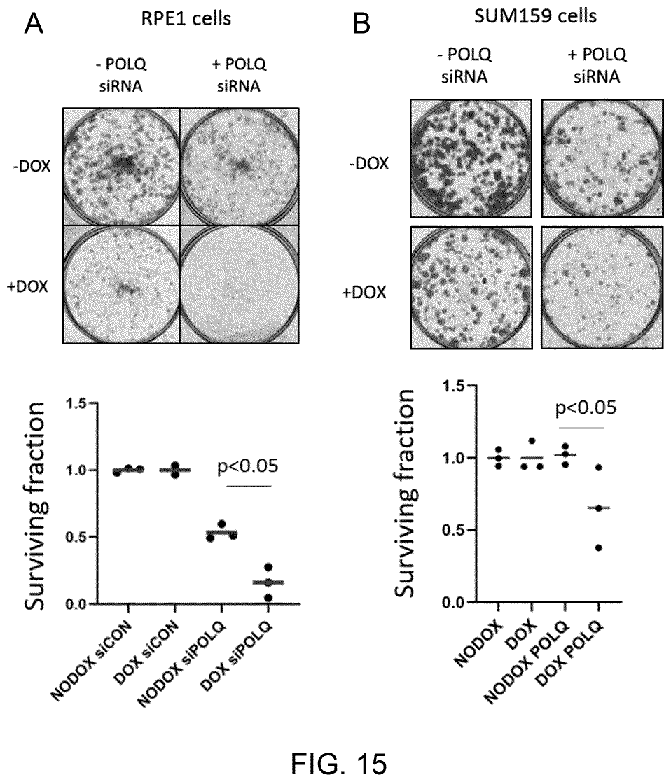

RPE1 (FIG. 15, A) or SUM159 (FIG. 15, B) cells engineered with dox inducible expression of HORMAD1 were transfected with NT (control) or POLQ siRNA, seeded at colony forming density and grown in the presence or absence of dox. Colonies were counted and surviving fractions for each condition was calculated.

In RPE1 cells, a significantly greater reduction in the surviving fraction of colonies following POLQ knockdown was observed in cells in which HORMAD1 expression was induced (+DOX), when compared to control cells to which no inducer was added. The data therefore demonstrate that HORMAD1-expressing cells have greater sensitivity to POLQ knockdown.

Similarly to the RPE-1 cell line, siRNA mediated POLQ knockdown resulted in a significant reduction in the surviving fraction of SUM159 cells expressing HORMAD1. POLQ knockdown did not result in a reduction in surviving fraction in the absence of the inducer. This suggests that the effect of modulating POLQ expression (and therefore activity) on cell survival is not only stronger, but specific to cells expressing HORMAD1.

### Dox-Induced Expression of HORMAD1 Increases Sensitivity to POLQ Inhibitors

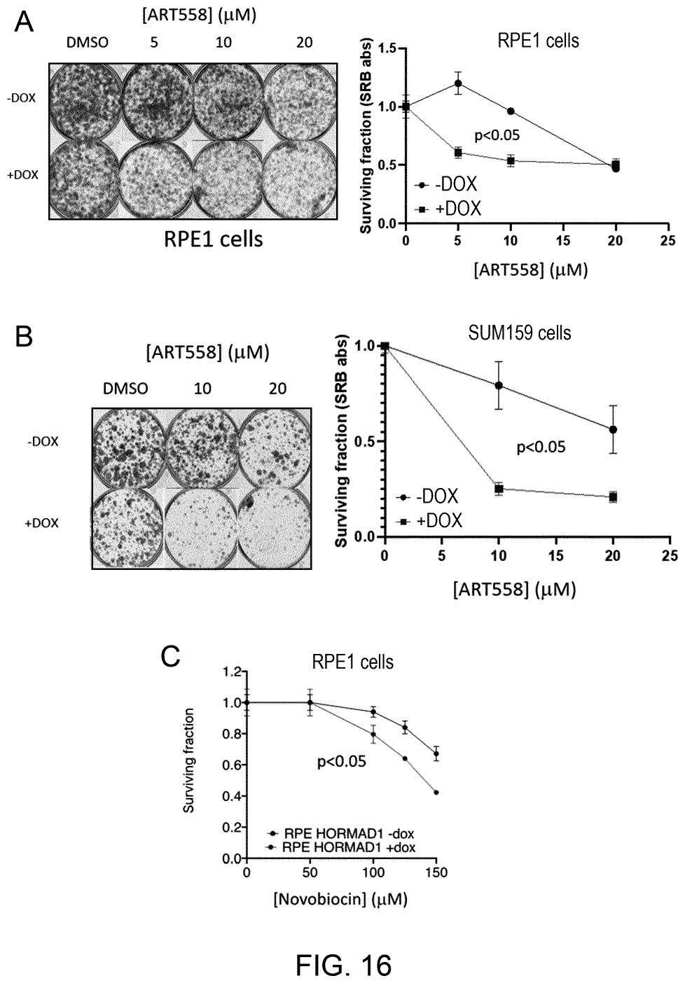

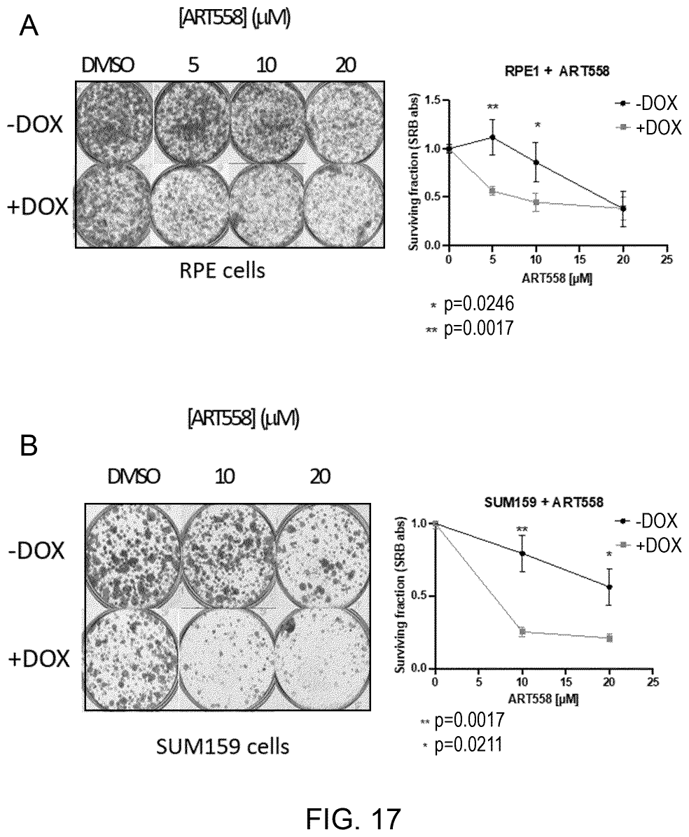

RPE1 (FIGS. 16-17, A) or SUM159 (FIGS. 16-17, B) cells engineered with dox inducible expression of HORMAD1 were seeded at colony forming density in the presence or absence of dox and treated with the indicated doses of ART558. Colonies were counted and surviving fractions for each condition were calculated.

In RPE1 cells expressing HORMAD1, a significant reduction in cell viability was observed in the presence of 5, 10, and 20 μM ART558. In contrast, in the absence of the inducer, no reduction in cell viability was seen below 20 μM ART558.

In SUM159 cells expressing HORMAD1, a significant reduction in cell viability was observed in the presence of both 10 and 20 μM ART558. Cells expressing HORMAD1 also showed significantly increased sensitivity to ART558 when compared to uninduced controls.

HORMAD1 negative RPE-1 cells engineered to have doxycycline inducible expression of HORMAD1 were then assessed for their sensitivity to the POLQ inhibitor Novobiocin. Cells were treated with the indicated doses of Novobiocin for 10 days and cell viability assessed by cell titre glow. Surviving fractions of triplicate wells are shown in FIG. 16, C.

At concentrations above 50 μM, Novobiocin treatment caused a significant and dose-dependent reduction in colony survival. Cells expressing HORMAD1 showed significantly increased sensitivity to Novobiocin when compared to uninduced controls, and the observed difference in colony survival between the two groups was also dose-dependent.

### CHK1 Inhibitor Sensitivity is Associated with HORMAD1 Expression

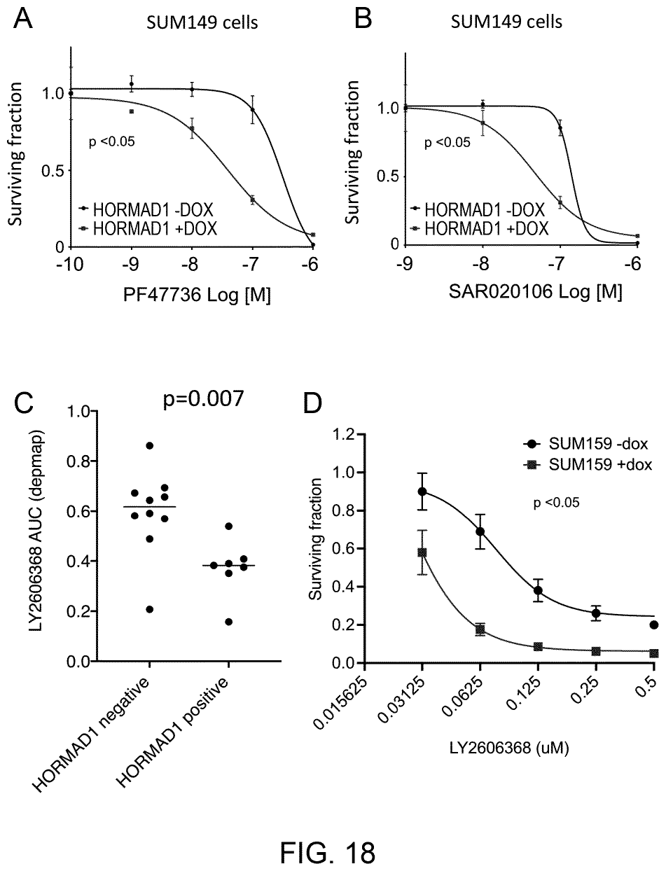

SUM149 cells (an inflammatory breast cancer cell line) with dox-inducible expression of HORMAD1 were seeded at colony forming density and treated with the indicated doses of the CHK1 inhibitors PF47736, SAR020106, and LY2606368. Colonies were counted, cell viability was assessed using Cell Titer Glow, and surviving fraction of triplicate wells are shown (FIGS. 18A, B and D). Cells expressing HORMAD1 showed significantly increased sensitivity to all three inhibitors when compared to uninduced controls. Data on the sensitivity of LY2606368 is available from depmap.org. Breast cancer cell lines were characterized as HORMAD1 negative and positive based on gene expression and the area under the curve (AUC) values for LY2606368 plotted. The HORMAD1 positive group of breast cancer cell lines has a lower mean AUC compared to the HORMAD1 negative group indicating increased sensitivity to LY2606368 in HORMAD1 positive breast cancer cell lines (FIG. 18C).

### HORMAD1 Knock-Down Reduces Sensitivity to POLQ Inhibitors

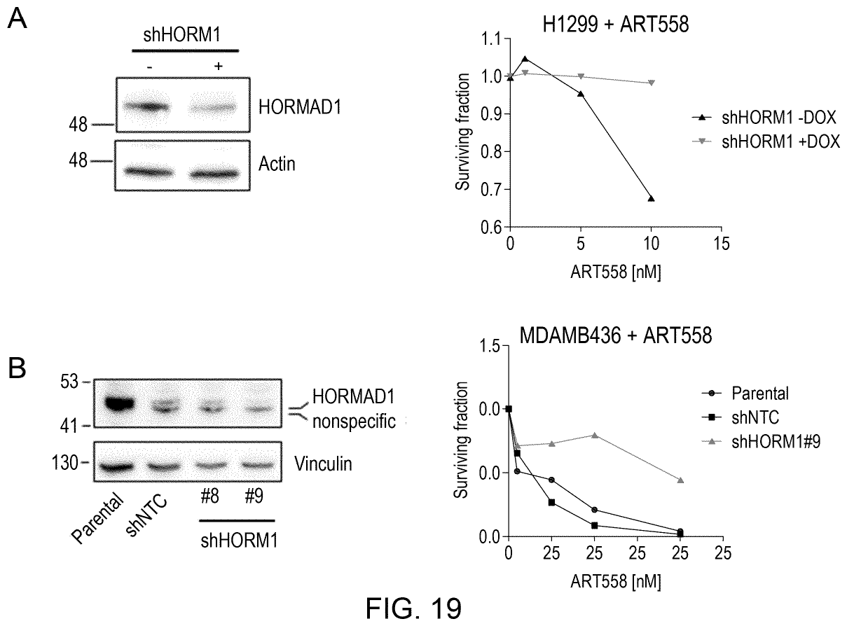

As shown in FIG. 19, dox-inducible knock down of HORMAD1 in H1299 (A) and constitutive knock down of HORMAD1 in MDA-MB-436 (B) decreases sensitivity to POLQ inhibitors.

In conclusion, the present invention has identified that HORMAD1 gene amplification, mRNA expression, or protein expression can be used as a cancer biomarker, for targeting the use of agents that generate DNA replication stress and/or inhibit pathways involved in DNA replication stress tolerance.

Since HORMAD1 can be readily detected in tumour samples, this data provides a pre-clinical rationale for the use of HORMAD1 expression as a predictive biomarker of sensitivity to agents that generate DNA replication stress and/or inhibit pathways involved in DNA replication stress tolerance. Furthermore, as shown in FIGS. 1, A-C, aberrant HORMAD1 expression is seen across a broader range of cancer types and tissues, and at higher frequency, than many biomarkers currently used for targeted cancer therapies, for example the BRCA1 mutation. HORMAD1 is therefore a particularly effective biomarker for targeting the use of agents that generate DNA replication stress and/or inhibit pathways involved in DNA replication stress tolerance.

### REFERENCES

Bass TE, Luzwick JW, Kavanaugh G, Carroll C, Dungrawala H, Glick GG, et al. ETAA1 acts at stalled replication forks to maintain genome integrity. Nat Cell Biol. 2016; 18(11): 1185-95.
Berti M, Vindigni A. Replication stress: getting back on track. Nat Struct Mol Biol. 2016;23(2): 103-9.
Brough R, Frankum JR, Sims D, Mackay A, Mendes-Pereira AM, Bajrami I, et al. Functional viability profiles of breast cancer. Cancer Discov. 2011;1(3):260-73.
Campbell J, Ryan CJ, Brough R, Bajrami I, Pemberton HN, Chong IY, et al. Large-Scale Profiling of Kinase Dependencies in Cancer Cell Lines. Cell Rep. 2016; 14(10):2490-501.
Futreal PA, Coin L, Marshall M, Down T, Hubbard T, Wooster R, et al. A census of human cancer genes. Nat Rev Cancer. 2004;4(3):177-83.
Gao Y, Kardos J, Yang Y, Tamir TY, Mutter-Rottmayer E, Weissman B, et al. The Cancer/Testes (CT) Antigen HORMAD1 promotes Homologous Recombinational DNA Repair and Radioresistance in Lung adenocarcinoma cells. Sci Rep. 2018;8(1): 15304.
Haahr P, Hoffmann S, Tollenaere MA, Ho T, Toledo LI, Mann M, et al. Activation of the ATR kinase by the RPA-binding protein ETAA1. Nat Cell Biol. 2016; 18(11):1196-207.
Halazonetis TD, Gorgoulis VG, Bartek J. An oncogene-induced DNA damage model for cancer development. Science. 2008;319(5868):1352-5.
Il'ina IV, Dyrkheeva NS, Zakharenko AL, Sidorenko AY, Li-Zhulanov NS, Korchagina DV, et al. Design, Synthesis, and Biological Investigation of Novel Classes of 3-Carene-Derived Potent Inhibitors of TDP1. Molecules. 2020;25(15).
Jones RM, Mortusewicz O, Afzal I, et al. Increased replication initiation and conflicts with transcription underlie Cyclin E-induced replication stress. Oncogene. 2013;32(32):3744-3753. doi:10.1038/onc.2012.387
Jones SE, Fleuren EDG, Frankum J, Konde A, Williamson CT, Krastev DB, et al. ATR Is a Therapeutic Target in Synovial Sarcoma. Cancer Res. 2017;77(24):7014-26.
Karnitz LM, Zou L. Molecular Pathways: Targeting ATR in Cancer Therapy. Clin Cancer Res. 2015;21(21):4780-5.
Kent T, Chandramouly G, McDevitt SM, Ozdemir AY, Pomerantz RT. Mechanism of microhomology-mediated end-joining promoted by human DNA polymerase θ. Nat Struct Mol Biol. 2015;22(3):230-237. doi:10.1038/nsmb.2961
Ketkar A, Maddukuri L, Penthala NR, Reed MR, Zafar MK, Crooks PA, et al. Inhibition of Human DNA Polymerases Eta and Kappa by Indole-Derived Molecules Occurs through Distinct Mechanisms. ACS Chem Biol. 2019;14(6):1337-51.
Kumagai A, Lee J, Yoo HY, Dunphy WG. TopBP1 activates the ATR-ATRIP complex. Cell. 2006;124(5):943-55.
Liu, K., Wang, Y., Zhu, Q., Li, P., Chen, J., Tang, Z., Shen, Y., Cheng, X., Lu, L. Y., and Liu, Y. (2020). Aberrantly expressed HORMAD1 disrupts nuclear localization of MCM8-MCM9 complex and compromises DNA mismatch repair in cancer cells. Cell death & disease 11, 519.
Lord CJ, McDonald S, Swift S, Turner NC, Ashworth A. A high-throughput RNA interference screen for DNA repair determinants of PARP inhibitor sensitivity. DNA Repair (Amst). 2008;7(12):2010-9.
Luo J, Solimini NL, Elledge SJ. Principles of cancer therapy: oncogene and non-oncogene addiction. Cell. 2009;136(5):823-37.
MacDougall CA, Byun TS, Van C, Yee MC, Cimprich KA. The structural determinants of checkpoint activation. Genes Dev. 2007;21(8): 898-903.
McDonald ER, 3rd, de Weck A, Schlabach MR, Billy E, Mavrakis KJ, Hoffman GR, et al. Project DRIVE: A Compendium of Cancer Dependencies and Synthetic Lethal Relationships Uncovered by Large-Scale, Deep RNAi Screening. Cell. 2017; 170(3):577-92 e10.
Meerbrey KL, Hu GA, Kessler JD, Roarty K, Li MZ, Fang JE, et al. The pINDUCER lentiviral toolkit for inducible RNA interference in vitro and in vivo. P Natl Acad Sci USA. 2011;108(9):3665-70.
Nichols BA, Oswald NW, McMillan EA, McGlynn K, Yan J, Kim MS, et al. HORMAD1 Is a Negative Prognostic Indicator in Lung Adenocarcinoma and Specifies Resistance to Oxidative and Genotoxic Stress. Cancer Res. 2018;78(21):6196-208.
Pommier Y, Huang SY, Gao R, Das BB, Murai J, Marchand C. Tyrosyl-DNA-phosphodiesterases (TDP1 and TDP2). DNA Repair (Amst). 2014;19:114-29.
Ryan CJ, Bajrami I, Lord CJ. Synthetic Lethality and Cancer—Penetrance as the Major Barrier. Trends Cancer. 2018;4(10):671-83.
Sale JE, Lehmann AR, Woodgate R. Y-family DNA polymerases and their role in tolerance of cellular DNA damage. Nat Rev Mol Cell Biol. 2012; 13(3):141-52.
Temprine K, Campbell NR, Huang R, Langdon EM, Simon-Vermot T, Mehta K, et al. Regulation of the error-prone DNA polymerase Polkappa by oncogenic signaling and its contribution to drug resistance. Sci Signal. 2020;13(629).
Tonzi P, Yin Y, Lee CWT, Rothenberg E, Huang TT. Translesion polymerase kappa-dependent DNA synthesis underlies replication fork recovery. Elife. 2018;7.
Uhlen, M., L. Fagerberg, B. M. Hallstrom, C. Lindskog, P. Oksvold, A. Mardinoglu, A. Sivertsson, et al. “Proteomics. Tissue-Based Map of the Human Proteome.” [In eng]. Science 347, no. 6220 (Jan. 23 2015): 1260419.
Uhlen, M., C. Zhang, S. Lee, E. Sjostedt, L. Fagerberg, G. Bidkhori, R. Benfeitas, et al. “A Pathology Atlas of the Human Cancer Transcriptome.” [In eng]. Science 357, no. 6352 (Aug. 18 2017).
Wagner SA, Oehler H, Voigt A, Dalic D, Freiwald A, Serve H, et al. ATR inhibition rewires cellular signaling networks induced by replication stress. Proteomics. 2016; 16(3):402-16.
Wang X, Tan Y, Cao X, Kim JA, Chen T, Hu Y, et al. Epigenetic activation of HORMAD1 in basal-like breast cancer: role in Rucaparib sensitivity. Oncotarget. 2018;9(53):30115-27.
Watkins J, Weekes D, Shah V, Gazinska P, Joshi S, Sidhu B, et al. Genomic Complexity Profiling Reveals That HORMAD1 Overexpression Contributes to Homologous Recombination Deficiency in Triple-Negative Breast Cancers. Cancer Discov. 2015;5(5):488-505.
Wojtaszek JL, Chatterjee N, Najeeb J, Ramos A, Lee M, Bian K, et al. A Small Molecule Targeting Mutagenic Translesion Synthesis Improves Chemotherapy. Cell. 2019; 178(1):152-9 e11.
Yamanaka K, Chatterjee N, Hemann MT, Walker GC. Inhibition of mutagenic translesion synthesis: A possible strategy for improving chemotherapy? PLoS Genet. 2017; 13(8):e1006842.
Yang W, Gao Y. Translesion and Repair DNA Polymerases: Diverse Structure and Mechanism. Annu Rev Biochem. 2018;87:239-61.
Yap TA, O'Carrigan B, Penney MS, Lim JS, Brown JS, de Miguel Luken MJ, et al. Phase I Trial of First-in-Class ATR Inhibitor M6620 (VX-970) as Monotherapy or in Combination With Carboplatin in Patients With Advanced Solid Tumors. J Clin Oncol. 2020: JCO1902404.
Zafar MK, Maddukuri L, Ketkar A, Penthala NR, Reed MR, Eddy S, et al. A Small-Molecule Inhibitor of Human DNA Polymerase eta Potentiates the Effects of Cisplatin in Tumor Cells. Biochemistry. 2018;57(7):1262-73.
Zeman MK, Cimprich KA. Causes and consequences of replication stress. Nat Cell Biol. 2014;16(1):2-9.
Zou L, Elledge SJ. Sensing DNA damage through ATRIP recognition of RPA-ssDNA complexes. Science. 2003;300(5625):1542-8.

1-19. (canceled)

20. A method of treating a patient with cancer, said method comprising:
a) determining whether said cancer, or a test sample from said patient, expresses HORMAD1; and, if so
b) administering to said patient an agent that generates DNA replication stress and/or inhibits pathways involved in DNA replication stress tolerance or a composition comprising said agent.

21. (canceled)

22. An in-vitro method for identifying an individual with cancer having suitability for treatment with an agent that generates DNA replication stress and/or inhibits pathways involved in DNA replication stress tolerance or a composition comprising said agent, said method comprising determining whether a cell sample from said individual expresses HORMAD1.

23. The method of claim 20, wherein the agent modulates the expression and/or activity of DNA polymerase theta.

24. The method of claim 20, wherein the agent is a DNA polymerase theta inhibitor.

25. The method of claim 20, wherein the DNA polymerase theta inhibitor is ART558, or a pharmaceutically acceptable salt thereof.

26. The method of claim 20, wherein the DNA polymerase inhibitor is Novobiocin, or a pharmaceutically acceptable salt thereof.

27. The method of claim 20, wherein the agent modulates the expression and/or activity of one or more translesion synthesis (TLS) polymerases.

28. The method of claim 20, wherein the one or more TLS polymerases are selected from REV1, POLH, POLK, and POLζ.

29. The method of claim 20, wherein POL comprises REV3L and REV7 subunits, and wherein modulating the expression and/or activity of POLζ comprises modulating the activity and/or expression of one or both of REV3L and REV7.

30. The method of claim 20, wherein the agent modulates the expression and/or activity of one or more cell-cycle checkpoint kinases, optionally ATR and/or CHK1.

31. The method of claim 20, wherein the agent modulates the expression and/or activity of TDP1.

32. The method of claim 20, wherein the agent modulates the expression and/or activity of BRIP1.

33. The method of claim 20, wherein the agent modulates the expression and/or activity of XRCC1.

34. The method of claim 20, wherein the cancer is a HORMAD1 positive cancer selected from breast cancers, such as triple negative (ER, PgR, and HER2 negative) and/or basal like breast cancers, leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.

35. A method of treating a patient with cancer, said method comprising:
a) determining whether said cancer or a test sample from the patient expresses HORMAD1; and, if so
b) administering to said patient a composition comprising an agent that generates DNA replication stress and/or inhibits pathways involved in DNA replication stress tolerance.

36. The method of claim 35, wherein the agent modulates the expression and/or activity of DNA polymerase theta or wherein the agent modulates the expression and/or activity of one or more translesion synthesis (TLS) polymerases.

37. The method of claim 35, wherein the one or more TLS polymerases are selected from REV1, POLH, POLK, and POLζ, wherein POLζ comprises REV3L and REV7 subunits, and wherein modulating the expression and/or activity of POL1 comprises modulating the activity and/or expression of one or both of REV3L and REV7.

38. The method of claim 35, wherein the agent modulates the expression and/or activity of one or more cell-cycle checkpoint kinases, optionally ATR and/or CHK1.

39. The method of claim 35, wherein the agent modulates the expression and/or activity of TDP1 or modulates the expression and/or activity of BRIP1 or modulates the expression and/or activity of XRCC1.

40. The method of claim 35, wherein the cancer is a HORMAD1 positive cancer selected from breast cancers, such as triple negative (ER, PgR, and HER2 negative) and/or basal like breast cancers, leukaemia, sarcomas, uveal melanomas, cholangiocarcinoma, melanomas, colorectal cancers, germ cell tumours of the testis and cancers of the bladder, cervix, oesophagus, head & neck, lung, ovary, pancreas, stomach, thyroid and uterus.
